(19) United States
(12) Patent Application Publication
(10) Pub. No.: US 2007/0116984 A1
(43) Pub. Date: May 24, 2007

(54) SPIRO-COMPOUND FOR ELECTROLUMINESCENT DISPLAY DEVICE AND ELECTROLUMINESCENT DISPLAY DEVICE COMPRISING THE SAME

(75) Inventors: Jong-Wook PARK, Seoul (KR); Ji-Hoon LEE, Chungju-si (KR); Cheol-Kyu CHOI, Yongin-si (KR); Kyoung-Soo KIM, Daejeon-si (KR); Sang-Do LEE, Yongin-si (KR)

Correspondence Address: SHERIDAN ROSS PC, 1560 BROADWAY, SUITE 1200, DENVER, CO, 80202, US

(73) Assignee: DOOSAN CORPORATION, Seoul (KR)

(21) Appl. No.: 11/534,098

(22) Filed: Sep. 21, 2006

(30) Foreign Application Priority Data
Sep. 21, 2005 (KR) 10-2005-0087802
Jul. 28, 2006 (KR) 10-2006-0071430

(51) Int. Cl. H01L 51/54; C09K 11/06

(52) U.S. Cl. 428/690; 428/917; 313/504; 313/506; 257/40; 257/E51.028; 257/E51.05; 546/15; 549/41; 528/377; 528/423; 528/403; 528/40

(57) ABSTRACT

The present invention relates to a spiro-compound for an electroluminescence display device and an electroluminescence display device including the same. More particularly, the present invention relates to a spiro-compound comprising at least one selected from the group consisting of a compound represented as the following Formulae 1 and 2 and an electroluminescence display device including the same: 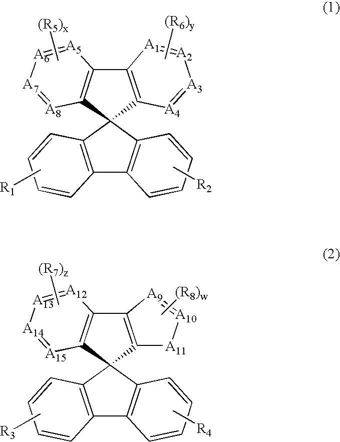 In the above Formulae 1 and 2, the definition of the substituents is the same as in the specification. The spiro-compounds represented by the above Formulae 1 and 2 are applicable to any one of a hole injection layer (HIL), a hole transport layer (HTL), an electroluminescent layer, an electron transport layer (ETL), and an electron injection layer (EIL) of the electroluminescence display device. The spiro-compound can realize various colors with low energy, emit blue light even at a low voltage, and have an advantage of excellently increasing luminance and luminous efficiency.

### BACKGROUND OF THE INVENTION

(a) Field of the Invention

The present invention relates to spiro-compounds for an electroluminescence display device and an electroluminescence display device including the same. More particularly, the present invention relates to spiro-based compounds applicable to any one of a hole injection layer (HIL), a hole transport layer (HTL), an electroluminescent layer (EML), an electron transport layer (ETL), and an electron injection layer (EIL), and a highly efficient organic electroluminescence display device including the same.

(b) Description of the Related Art

These days, as development within the information and communication industry is accelerated, higher performance display devices are required. Such display devices may be classified into luminescence types and non-luminescence types.

For the former devices, a Cathode Ray Tube (CRT), an Electroluminescence Display (ELD), a Light Emitting Diode (LED), a Plasma Display Panel (PDP), etc., are exemplified. For the latter devices, a Liquid Crystal Display (LCD), etc., are exemplified.

The luminescence type and non-luminescence type display devices have basic characteristics such as working voltage, consumption power, brightness, contrast, response time, lifetime, and color display, etc. However, liquid crystal display devices, which have largely been used until now, have problems in terms of response time, contrast, and viewing angle among the basic characteristics described above.

Displays using a luminescence diode are expected as the next generation display devices that can solve the problems of liquid crystal displays since they have a short response time and do not require a backlight due to having self-luminescence properties, and they also have improved brightness, etc.

An electroluminescence diode has difficulties in application to a large area electroluminescence display device because an inorganic material with crystalline form is mainly used. Furthermore, in the case of an electroluminescence display device using an inorganic material, there are disadvantages that more than 200 V of driving voltage is required and it is expensive. Active research on electroluminescence display devices including an organic material has been undertaken since the Eastman Kodak Company disclosed a device made from a material having a π-conjugated structure in 1987. In the case of an organic material, there are advantages that a synthetic pathway is relatively simpler and various forms of materials can be synthesized, and thus color tuning is possible. On the contrary, the organic material has disadvantages in that crystallization by heat occurs due to low mechanical strength.

Organic materials used in an electroluminescence display device are classified into low molecular organic materials and high molecular organic materials. For low molecular organic materials diamine, diamine derivatives such as N,N′-bis-(4-methylphenyl)-N,N′-bis(phenyl)benzidine (TPD), etc., derivatives of perylene tetracarboxylic acid, oxadiazole derivatives, 1,1,4,4-tetraphenyl-1,3-butadiene (TPB), etc., are exemplified.

In 1990, the Cambridge group reported that poly(1,4-phenylenevinylene) (PPV) π-conjugated polymers emit lights when electricity is applied thereto. Since then, there are active researches for polymers used in an electroluminescence display device.

π-conjugated polymers have an alternate structure of single bonds (σ-bonds) and double bonds (π-bond), and include π-electrons that are not locally distributed and are free to move along bond chains. Since π-conjugated polymers have such semiconductor characteristics, the polymers can be obtained through molecular designs to emit lights at all visible areas corresponding to HOMO-LUMO band-gaps when they are applied to an electroluminescent layer of an electroluminescence display device. Since the polymers are easily formed in a thin layer using spin coating or a printing method, a device manufacturing process becomes easy and the costs low. They also have merits to provide a thin layer having excellent mechanical properties due to their high glass transition temperature. However, the polymers may have defects to facilitate deterioration in a molecular chain depending on their synthesizing methods, and are difficult to purify to obtain high purity products.

### SUMMARY OF THE INVENTION

One embodiment of the present invention provides a spiro-compound with excellent light emitting characteristics for an electroluminescence display device.

The spiro-compound is a spiro-based compound including a heteroatom, which can be applied to any of a hole injection layer (HIL), a hole transport layer (HTL), an electroluminescent layer, an electron transport layer (ETL), and an electron injection layer (EIL) of an electroluminescence display device. Another embodiment of the present invention provides an electroluminescence display device that includes the spiro-compound.

According to an embodiment of the present invention, a spiro-compound for an electroluminescence display device is provided that is at least one selected from the group consisting of compounds represented by the following Formulae 1 and 2, an oligomer thereof, and a polymer thereof.

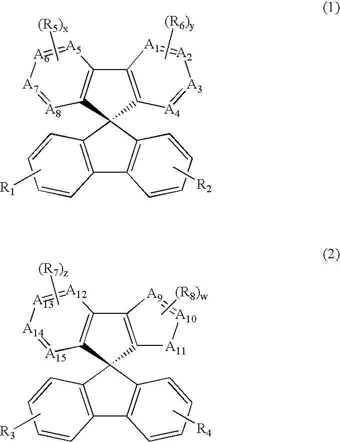

Wherein, in the above Formulae, A₁ to A₁₅ are independently an element selected from the group consisting of C, N, O, S, and Si, and at least one of A₁ to A₈ and at least one A₉ to A₁₅ are not carbon (C),

R₁ to R₄ are independently selected from the group consisting of hydrogen, deuterium, halogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, and a substituted or unsubstituted heteroaryl,

R₅ to R₈ are selected from the group consisting of hydrogen, deuterium, halogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, a substituted or unsubstituted heteroaryl, —CN, —NO₂, a substituted or unsubstituted fluoroalkyl, —SiR₉R₁₀R₁₁, —NR₁₂R₁₃, and —CR₁₄═CR₁₅—R₁₆,

R₉ to R₁₆ are independently selected from the group consisting of hydrogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, and a substituted or unsubstituted heteroaryl, and

x, y, z, and w are integers ranging from 0 to 4.

According to another embodiment of the present invention, an electroluminescence display device is provided that includes: a substrate; an anode; a hole injection layer (HIL); a hole transport layer (HTL); an electroluminescent layer; an electron transport layer (ETL); an electron injection layer (EIL); and a cathode. At least one of the hole injection layer (HIL), the hole transport layer (HTL), the electroluminescent layer, the electron transport layer (ETL), and the electron injection layer (EIL) include the above spiro-compound.

### BRIEF DESCRIPTION OF THE DRAWINGS

FIG. 1 is a schematic cross-sectional view of an organic electroluminescence display device according to a first embodiment of the present invention.

FIG. 2 is a schematic cross-sectional view of an electroluminescence display device according to a second embodiment of the present invention that further adds a buffer layer to the device of the first embodiment.

FIG. 3 is a schematic cross-sectional view of an electroluminescence display device according to a third embodiment of the present invention that further adds an electron injection layer (EIL) to the device of the first embodiment.

FIG. 4 shows proton nuclear magnetic resonance (¹H-NMR) spectrum of a dibromo compound 7 according to Example 1 of the present invention.

FIG. 5 shows proton nuclear magnetic resonance (¹H-NMR) spectrum of a dibromo compound 11 according to Example 2 of the present invention.

FIG. 6 shows proton nuclear magnetic resonance (¹H-NMR) spectrum of a dibromo compound 13 according to Example 4 of the present invention.

FIG. 7 shows proton nuclear magnetic resonance (¹H-NMR) spectrum of a polymer 1 according to Example 25 of the present invention.

FIG. 8 shows proton nuclear magnetic resonance (¹H-NMR) spectrum of a polymer 2 according to Example 26 of the present invention.

FIG. 9 shows TGA analysis data of a compound 12 according to Example 3 of the present invention.

FIG. 10 shows TGA analysis data of a compound 13 according to Example 4 of the present invention.

FIG. 11 shows TGA analysis data of a polymer 1 according to Example 25 of the present invention.

FIG. 12 shows TGA analysis data of a polymer 2 according to Example 26 of the present invention.

FIG. 13 is a graph showing UV-visible ray absorption (UV-vis) spectrum of a compound 12 according to Example 3 of the present invention.

FIG. 14 is a graph showing a PL (photoluminescence) spectrum of a compound 12 according to Example 3 of the present invention.

FIG. 15 is a graph showing UV-visible ray absorption (UV-vis) spectrum of a compound 13 according to Example 4 of the present invention.

FIG. 16 is a graph showing a PL (photoluminescence) spectrum of a compound 13 according to Example 4 of the present invention. FIG. 17 shows UV-vis spectrum and PL (photoluminescence) spectrum of a polymer 1 according to Example 25.

FIG. 18 is a graph showing electroluminescence characteristics (current-voltage) of a compound 12 according to Example 3 of the present invention.

FIG. 19 is a graph showing electroluminescence characteristics (voltage-brightness) of a compound 12 according to Example 3 of the present invention.

FIG. 20 is a graph showing an EL spectrum of a compound 12 according to Example 3 of the present invention.

FIGS. 21 to 23 show light-emitting characteristics (for example, current-voltage, voltage-luminance, and current-luminance) of the polymer 1 according to Example 25.

FIG. 24 shows luminance efficiency characteristics of the polymer 1 according to Example 25 depending on a current increase.

FIG. 25 is a graph showing EL spectrum of the polymer 1 according to Example 25 of the present invention.

FIG. 26 is a graph showing EL spectrum of the polymer 3 according to Example 27 of the present invention.

### DETAILED DESCRIPTION OF THE INVENTION

Exemplary embodiments of the present invention will hereinafter be described in detail with reference to the accompanying drawings.

The present invention makes up for drawbacks of convential low molecule polymers while including their merits, and thereby, provides an electro-chromophore material that can be easily purified, has no molecule drawback, and can form a thin membrane by using a soluble solvent regardless of small molecular weight.

The electro-chromophore material is structurally based on spiro-based compounds including a heteroatom, but is still new because it includes various substitutes, and thereby, may be easily used in a vacuum or wet process spiro-compound.

According to the embodiments of the present invention, a spiro-compound for an electroluminescence display device is at least one selected from the group consisting of compounds represented by the following Formulae 1 and 2, an oligomer thereof, and a polymer thereof.

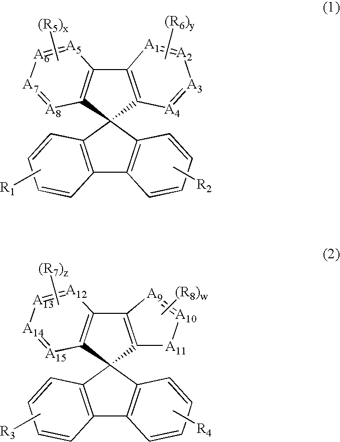

Wherein, in the above Formulae, A₁ to A₁₅ are independently an element selected from the group consisting of C, N, O, S, and Si, and at least one of A₁ to A₈ and at least one A₉ to A₁₅ are not carbon (C). According to one embodiment, in the Chemical Formula 1, at least one of A₁ to A₄ and at least one of A₅ to A₈ are selected from the group consisting of N, O, S, and Si, and in the Chemical Formula 2, at least one of A₉ to A₁₁ and at least one of A₁₂ to A₁₅ are selected from the group consisting of N, O, S, and Si. At least one of A₁ to A₁₅ is a heteroatom, and 1 to 4 of A₁ to A₁₅ is more preferably a heteroatom.

In the above Formulae 1 and 2, R₁ to R₄ are independently selected from the group consisting of hydrogen, deuterium, halogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, and a substituted or unsubstituted heteroaryl.

R₅ to R₈ are selected from the group consisting of hydrogen, deuterium, halogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, a substituted or unsubstituted heteroaryl, —CN, —NO₂, a substituted or unsubstituted fluoroalkyl, —SiR₉R₁₀R₁₁, —NR₁₂R₁₃, and —CR₁₄═CR₁₅—R₁₆.

R₉ to R₁₆ are independently selected from the group consisting of hydrogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, and a substituted or unsubstituted heteroaryl.

The halogen of R₅ to R₈ may be selected from the group consisting of F, Cl, Br, and I,

In R₁ to R₁₆, the alkyl may be a C1 to C12 alkyl, preferably a C1 to C8 lower alkyl; the cycloalkyl may be a C3 to C12 cycloalkyl, and preferably a C5 to C8 cycloalkyl; the alkenyl may be a C2 to C8 alkenyl, and preferably a C2 to C4 alkenyl; the alkynyl may be a C2 to C4 alkynyl, and preferably a C2 to C4 alkynyl; the alkoxy may be a C1 to C12 alkoxy, and preferably C1 to C8 alkoxy; the aryl may be C4 to C30 aryl, and preferably C4 to C20 aryl; and the heteroaryl may be a C4 to C30 heteroaryl including an aromatic ring including a 1 to 3 heteroatom of N, S, P, Si, or O, and preferably a C4 to C20 heteroaryl including an aromatic ring including a 1 to 3 heteroatom of N, S, P, Si, or O.

In the Chemical Formulae 1 and 2, the term “substituted” means at least one hydrogen is substituted with an alkyl, a cycloalkyl, an alkoxy, an alkenyl, an alkynyl, an aryl, a heteroaryl, a halogen such as F, Cl, Br, or I, aliphatic amine, aromatic amine, or an aryloxy.

In the Chemical Formulae 1 and 2, the heteroaryl is preferably substituted or unsubstituted carbazole, substituted or unsubstituted phenothiazine, substituted or unsubstituted phenoxazine, substituted or unsubstituted phenoxathin, substituted or unsubstituted acridine, substituted or unsubstituted phenazasiline, or substituted or unsubstituted 9-aza-10-germa-anthracene.

Since the spiro-compound has an excellent thermal characteristic and a three dimensional structure, it has small interaction among molecules, and thereby, excellent characteristics in terms of luminescent stability and the like. Therefore, it can be applied to anything of a hole injection layer (HIL), a hole transport layer (HTL), an electroluminescent layer, an electron transport layer (ETL), and an electron injection layer (EIL) of an electroluminescence (EL) device. In particular, since it has an excellent luminescent characteristic, it can be used as a host or a dopant of an electroluminescent layer.

However, the present invention may even include an oligomer, a homopolymer, or a copolymer prepared by using a compound represented in the above Chemical Formulae 1 or 2 as a monomer. The oligomer or polymer including a monomer of the Chemical Formulae 1 and 2 are represented as the following Formulae 3 and 4:

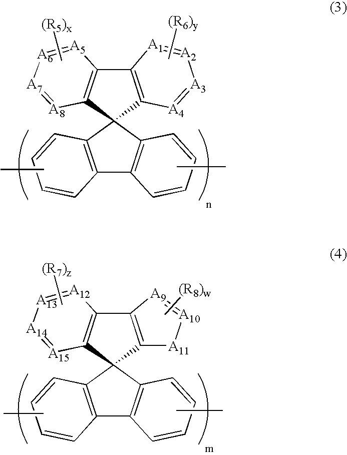

In the above Chemical Formulae 3 and 4, A₁ to A₈, R₅, and R₆ are the same as in the above Formula 1, and n and m may be in a range of 1 to 10000 and preferably, in a range of 1 to 2000. As for the oligomer, n and m may be in a range of 1 to 10, while as for the polymer, n and m may be in a range of 10 to 2000.

The oligomer or copolymer can be prepared through a solution polymerization by using a compound of Chemical Formulae 1 or 2 as a monomer and Ni (O), Pd (O), or the like, as a metal catalyst. The catalyst may include Ni(COD)₂ [bis(1,5-cyclooctadiene)nickel 0], Pd(Ph₃)₄ [tetrakis(triphenylphosphine)palladium 0], PdCl₂ [palladium(II) chloride], FeCl₃ [Iron(III) chloride], and the like.

When a compound of the above Chemical Formula 1 or 2 is polymerized, another compound of the following Chemical Formulae 5 and 6 can also be polymerized together. In general, the polymerization method may include Yamamoto or Suzuki polymerization.

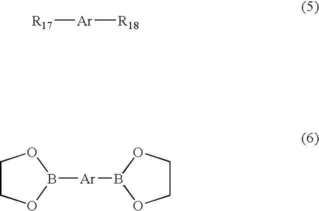

In the above formulae, Ar may include a substituted or an unsubstituted aromatic group or a heteroaromatic group including more than one hetero atom in an aromatic ring. R₁₇ and R₁₈ as a reactive functional group may independently include halogen, borate, boronic acid (—B(OH)₂), and OTf. In addition, R₁₇ and R₁₈ may be selected from the group consisting of hydrogen, an unsubstituted linear or branched alkyl group, a cyclo alkyl group, an alkenyl group, an alkynyl group, an alkoxy group, an aryl group, and a hetero aryl group. Their number of a carbon can be defined as illustrated above. When R₁₇ and R₁₈ are not a reactive functional group, an oxidant or a reducing agent can be added for the polymerization.

The aromatic group may have the number of a carbon ranging 4 to 30 and preferably, 4 to 20. The heteroaromatic group may have the number of a carbon ranging 4 to 14. The aromatic group and the heteroaromatic group can have more than one substitute such as an alkyl group having the number of a carbon ranging 1 to 12, an alkoxy group, or an amine group. The Ar may be illustrated as follows.

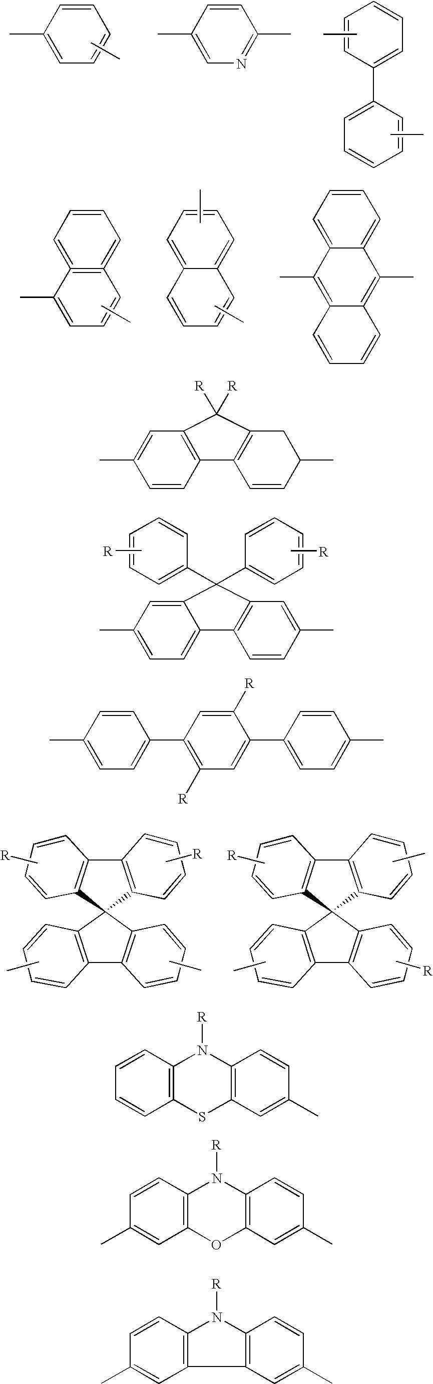

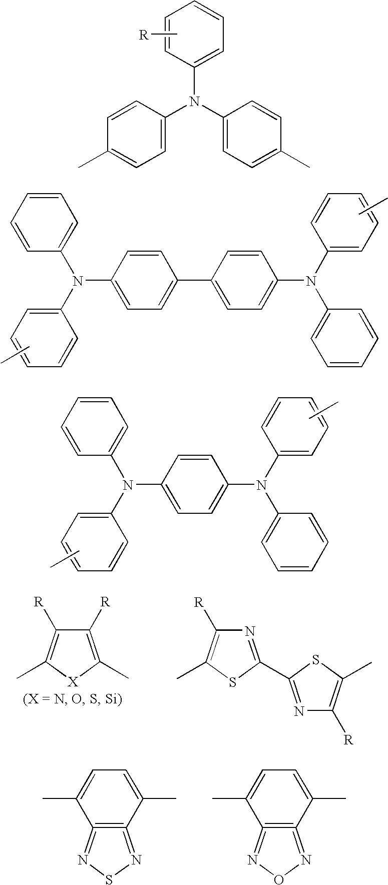

In the above formulae, R is respectively a hydrogen atom, a branch- or cyclic-type alkyl group or alkoxy group, and an aromatic group having the number of a carbon ranging 4 to 20, and preferably, 4 to 14. The aromatic group can have a substitute selected from the group consisting of an alkyl group having the number of a carbon ranging 1 to 12, an alkoxy group, or an amine group. X is selected from the group consisting of N, O, S, and Si.

At least either of a monomer of Chemical Formula 1 or 2 and a monomer of Chemical Formula 5 or 6 may be regulated to have a mole ratio in a range of 1:0.01 to 100, and preferably in a range of 1:0.03 to 10.

According to the embodiment of the present invention, a spiro-compound is applied between an anode for inserting a hole made of indium tinoxide (ITO) with a big work function and a cathode for inserting an electron made of a metal with various work functions such as aluminum, lithiumfluoride/aluminum, lithiumfluoride/calcium, bariumfluoride/calcium, copper, silver, calcium, gold, magnesium, and the like, an alloy of magnesium and silver, and an alloy of aluminum and lithium.

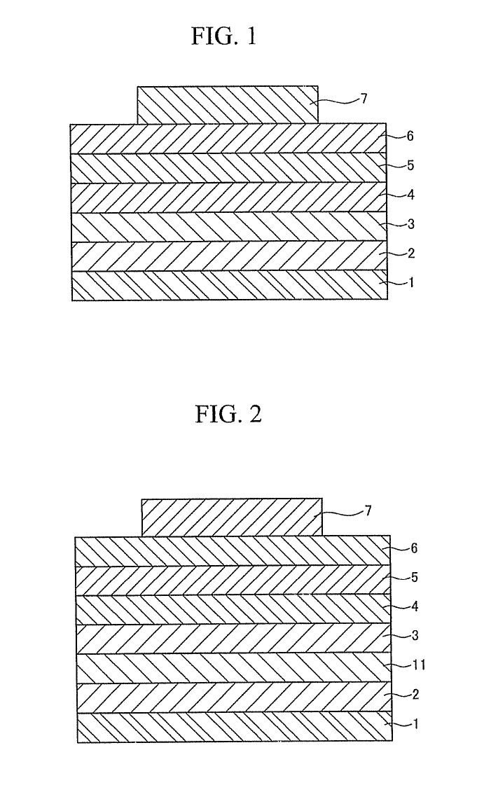

FIG. 1 is a cross-sectional view showing an electroluminescence display device according to one embodiment. Referring to FIG. 1, an electroluminescence display device of the present invention includes an anode 2, a hole injection layer (HIL) 3, a hole transport layer (HTL) 4, an electroluminescent layer 5, an electron transport layer (ETL) 6, and a cathode 7, which are sequentially stacked on a substrate 1. At least one of the hole injection layer (HIL) 3, the hole transport layer (HTL) 4, the electroluminescent layer 5, the electron transport layer (ETL) 6, and the cathode 7 includes the spiro-compound according to the present invention.

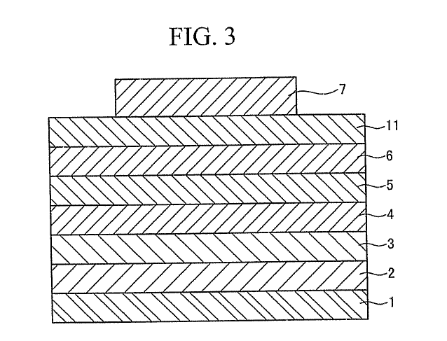

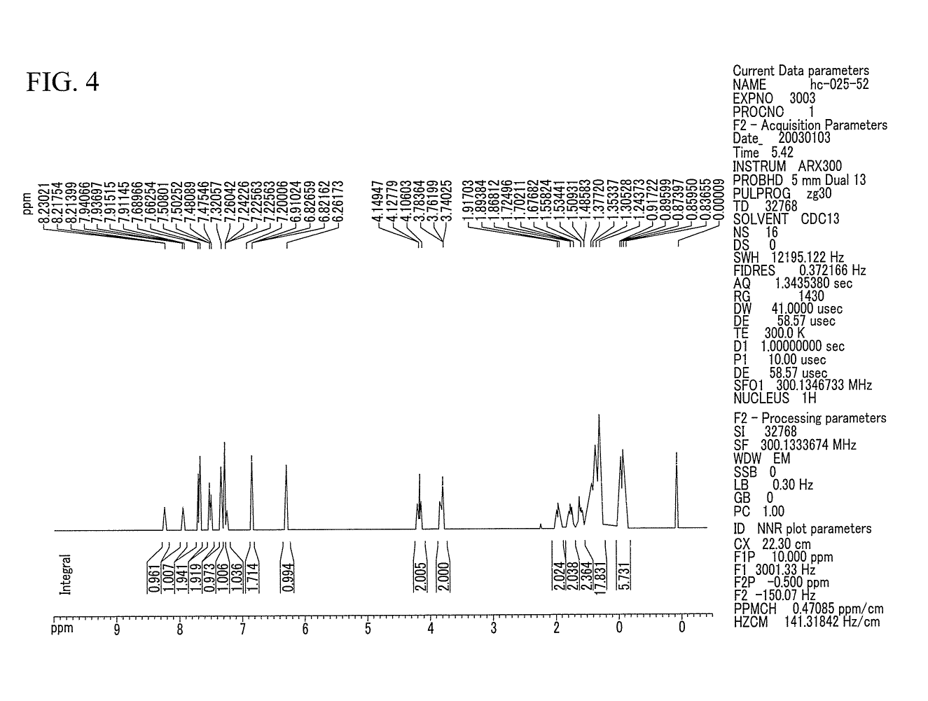

As shown in FIG. 2, an electroluminescence display device of the present invention may further include a buffer layer 11 between an anode 2 and a hole injection layer (HIL) 3.

In addition, the electroluminescence display device may include an electron injection layer (EIL) 12 between an electron transport layer (ETL) 6 and a cathode 7.

The substrate 1 includes a material, such as a glass, a plastic, quartz, a ceramic, or silicon, that has transparency, a flat-surface, and water-repellency, and is easy to handle, but is not limited thereto.

The anode 2 has a function of injecting holes, and includes an anode material having a large work function. The anode material may include transparent and highly conductive indium tin oxide (ITO), indium zinc oxide (IZO), tin oxide (SnO₂), zinc oxide (ZnO), and so on.

The buffer layer 11 exists to compensate the surface of the anode 2 and helps the injection and flow of the holes. Materials used as the buffer are exemplified by a conductive polymer material such as doped polyaniline (PANI) doped polyethylenedeoxythiopene (PEDOT), and low molecular materials such as alpha-copper ferrocyanine (CuPc). A thin film having a thickness from 20 nm to 150 nm was formed by spin coating PANI and PEDOT. Alternatively, a thin film having thickness from 20 nm to 100 nm might be formed by vacuum-deposition of alpha-CuPc. The above description for the buffer layer 11 refers to one embodiment, but is not limited to as above described.

The hole injection layer (HIL) 3 is formed on the anode 2 or the buffer layer 11 by coating a hole injection material using vacuum heat deposition, or a spin coating method. In the case of a low molecular electroluminescence display device, examples of the hole injection material are not particularly limited, but copper ferrocyanine (CuPc) or starburst-type amine such as 4,4′,4″-tris-(N-carbazolyl)-triphenyl amine (TCTA), 4,4′,4″-tris(3-methylphenylphenylamino)triphenyl amine (m-MTDATA), 1,3,5-tris[4-(3-methylphenylphenylamino)phenyl]benzene (m-MTDAPB), and so on, can be used.

The hole transport layer (HTL) 4 may be formed on the hole injection layer 3 using vacuum heat decomposition or spin coating of hole transport materials. The method of forming the hole transport layer is not limited thereto. The hole transport material is not limited to specific materials, and may be a material generally used in an electroluminescence display device. In the case of a low molecular electroluminescence display device, the hole transport material may be selected from the group consisting of N,N′-bis(3-methylphenyl)-N,N′-diphenyl-[1,1-biphenyl]-4,4′-diamine (TPD), N,N′-di(naphthalene-1-yl)-N,N′-diphenyl benzidine and N,N′-bis(naphthalene-1-yl)-N,N′-diphenyl-benzidine (-NPB). In the case of a polymer electroluminescence display device, a doped PEDOT may simultaneously be included in the hole injection layer (HIL) 3 and the hole transport layer (HTL) 4.

An electroluminescent layer (EML) 5 is formed on the hole transport layer 4 using vacuum heat deposition or spin coating of an electroluminescence material.

The electron transport layer (ETL) 6 formed on the electroluminescent layer 5 using vacuum deposition or spin coating. The electron transport layer may include at least one selected from the group consisting of aluminum tris(8-hydroxyquinoline)(Alq3), and 2-(4′-bisphenyl)-5-(4″-t-butylphenyl)-1,3,4-oxadiazole (t-Bu-PBD). The method of forming the electron transport layer and electron transport material are not limited to specific examples.

The electron injection layer (EIL) 12 may optionally be formed on the electron transport layer 6

The electron injection layer (EIL) may include generally used materials in an electroluminescence display device, and for example may be selected from the group consisting of LiF, BaF₂, NaCl, CsF, Li₂O, and BaO.

A cathode is formed on the electron transport layer (ETL) 6 or electron injection layer (EIL) 12 by coating a cathode material using vacuum heat deposition. The cathode may include a metal such as lithium (Li), magnesium (Mg), aluminum (Al), aluminum-lithium (Al—Li), calcium (Ca), magnesium-indium (Mg—In), magnesium-silver (Mg—Ag), and so on. A front electroluminescence display device may be obtained by using a transparent conductive material such as at least one selected from the group consisting of ITO and IZO to form a light-transmitting cathode.

The spiro-compound selected from the group consisting of the compound represented by the above Formulae 1 and 2, an oligomer and a polymer thereof are applicable to any one of a hole injection layer (HIL) 3, a hole transport layer (HTL) 4, an electroluminescent layer 5, an electron transport layer (ETL) 6, and an electron injection layer (EIL) 12 of the electroluminescence display device. According to preferred embodiment, the spiro-compound selected from the group consisting of the compound represented by the above Formula 1 or 2, an oligomer and a polymer thereof is applied to an electroluminescent layer 5.

When the spiro-compounds are applied to an electroluminescent layer 5, the spiro-compounds of the present invention can be used along with another chromophore material, a dopant, or another chromophore material and a dopant. When the spiro-compounds are used with another chromophore material, the spiro-compounds act as a dopant. When the spiro-compounds are used with a dopant, the spiro-compounds act as an electroluminescent host. When the spiro-compounds are used with another chromophore material and a dopant, they act as both host and dopant.

The chromophore material used with the spiro-compound has no particular limit and includes any chromophore materials. According to the preferred embodiment, it may be selected from the group consisting of aluminum tris(8-hydroxyquinilone) (Alq3) for emitting green, red, or yellow, 4,4′-bis(carbazole-9-yl)biphenyl (CBP) for emitting blue, 4′-bis(2,2-diphenyl-ethene-1-yl)-diphenyl (DPVBi), 4,4″-bis(2,2-diphenylvinyl-1-yl)-p-terphenylene (DPVTP), Spiro-DPVBi, and the like, depending on a dopant.

A dopant included in the electroluminescent layer 5 may include an organic compound with a conjugated double bond that has smaller energy gap than the spiro-compound as a material for being doped, and thereby, the dopant has a smaller maximum wavelength than the spiro-compound, transfers energy better, and has good chromophore characteristics.

A polymer electro-luminescence system can optimize luminescent characteristics such as luminescent color, efficiency, and operation voltage decrease by physically mixing at least 1 to 2 or at most 4 to 5 polymers. However, these blending systems can fundamentally optimize performance through copolymerization, which is a chemical bond, due to deteriorated durability of a thin polymer film such as phase separation and the like.

As for a low molecular electroluminescence display device, at least one compound selected from the group consisting of dicarbazolyl azobenzene (DCAB), fluorenyl diacetylene (FDA), perylene, carbazole, carbazole derivatives, coumarine compounds, and 4-(dicyanomethylene)-2-methyl-6-(1,1,7,7-tetramethyljulodinyl-9-enyl)-4H-pyran (DCJT) may be used as a dopant.

The dicarbazolyl azobenzene (DCAB) is represented as the following Formula 3, the fluorenyidiacetylene (FDA) as the following Formula 4, the perylene as the following Formula 5, the 4-(dicyanomethylene)-2-methyl-6-(1,1,7,7-tetramethyljulodinyl-9-enyl)-4H-pyran as the following Formula 6, and the coumarin-based compound as the following Formula 7 (Exciton Co.).

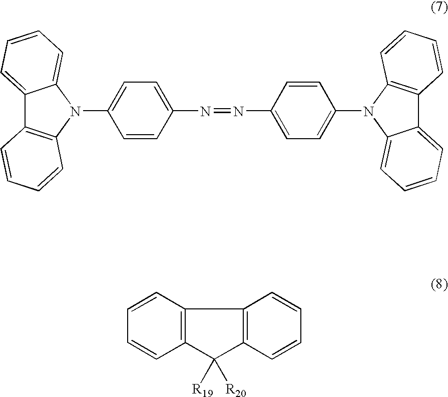

Wherein, in the above formulae, R₁₉ and R₂₀ are independently selected from the group consisting of hydrogen, an alkyl, an aryl, a cycloalkyl, and an acetyl.

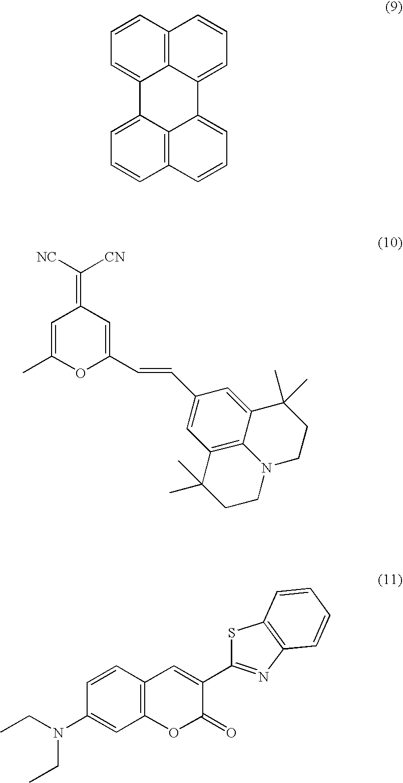

The dopants may have more than one substituent to obtain desirable properties, such as crystallization degree, thermal stability, and solubility.

Dicarbazolyl azobenzene (DCAB), fluorenyl diacetylene (FDA), perylene, carbazole and carbazole derivatives serve as blue dopants, coumarines compounds as green dopants, and 4-(dicyanomethylene)-2-methyl-6-(1,1,7,7-tetramethyljulodinyl-9-enyl)-4H-pyran serves as a red dopant.

The amount of the dopants is preferably 0.1 to 30% by weight, more preferably 5 to 30% by weight, and most preferably 5 to 10% by weight, based on the total weight of the chromophore material and dopants in an electroluminescent layer. Within this range, excellent electroluminescent properties can be obtained.

Hereinafter, preferred embodiments of the present invention will be described. However, these are presented only for better understanding of the present invention, and the present invention is not limited thereto.

### EXAMPLE 1

### Synthesis of Dibromo Compound 7

A dibromo compound 7 was synthesized according to the following Reaction Scheme 1.

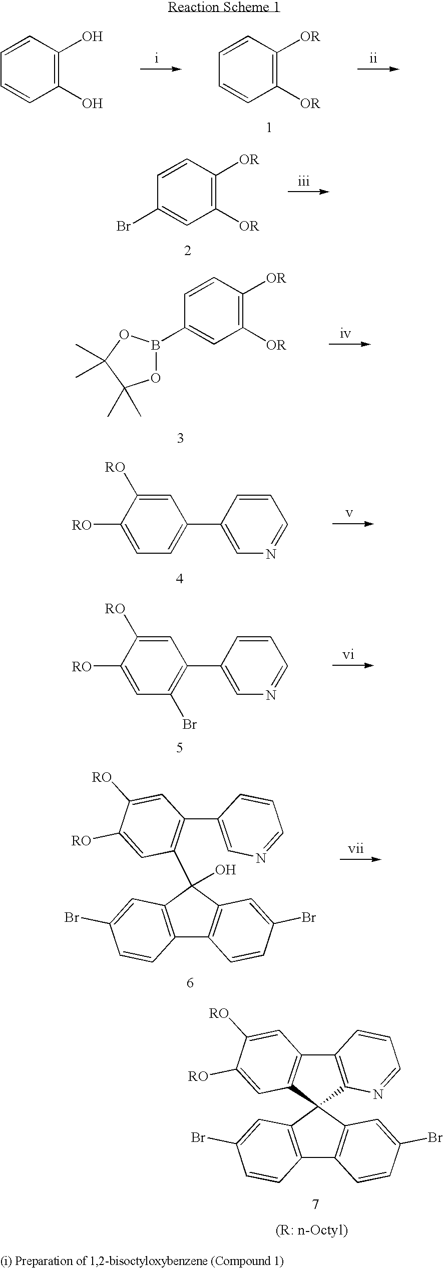

19 g of catechol was dissolved in 200 ml of acetonitrile, and then, 2.5 eq of 1-bromooctane, 2.5 eq of K₂CO₃, and 0.1 eq of KI were added thereto. The resulting mixture was heated and refluxed for 24 hours.

When the reaction was complete, the reactant was filtered to get an organic layer. Then, the filtered organic layer was concentrated under a reduced pressure. Then, the residue gained from the reduced pressure concentration was dissolved in 200 ml of ethylether. The gained organic layer was washed with 100 ml of water and salt-saturated water, so that it could be clearly separated. Then, it was dehydrated with 20 g of MgSO₄, and the remaining solution was concentrated under a reduced pressure, gaining 57.17 g of a white-solid compound 1 (yield=99%). The compound 1 was examined regarding its structure through a ¹H-NMR.

### (ii) Preparation of 4-bromo-1,2-bis-octyloxybenzene (Compound 2)

57.17 g of compound 1 was dissolved in 400 ml of methylene chloride. Separately, 1.1 eq of N-bromosuccinimide (NBS) was dissolved in 100 ml of N,N-dimethylformamide (DMF) at 0° C. Then, this solution was added in a dropwise fashion to the compound 1 solution. The mixture solution was heated up to room temperature and then reacted for 2 hours. When the reaction was complete, the reacting solution was twice washed with 200 ml of water. Then, the organic layer was washed with a Na₂S₂O₃.5H₂O solution, a NaHCO₃ saturated solution, and brine in order, and thereafter, treated with MgSO₄ and filtered. The solvent was concentrated under a reduced pressure, gaining 69.01 of a compound 2 (yield=98%). The produced compound 2 was examined regarding its structure through a ¹H-NMR.

### (iii) Preparation of 1-(3,4-bisoctyloxyphenyl)-3,3,4,4-tetramethyl-borolane (Compound 3)

69.03 g of a compound 2 was dissolved into 500 ml of anhydrous tetrahydrofuran (THF) in a 2 L-flask, and 1.2 eq of n-BuLi was added thereto in a dropwise fashion at 78° C. Then, the resulting mixture was agitated for ten minutes, and thereafter, 1.1 eq of 2-isoproxy-4,4,5,5-tetramethyl-1,3,2-dioxaborolane was slowly added thereto at the same temperature.

When the reaction was complete after agitating it for one hour, 300 ml of ethyl acetate and 300 ml of water were added thereto to separate an organic layer. The organic layer was washed with 150 ml of a saturated NaHCO₃ solution and 150 ml of salt water, and thereafter treated with MgSO₄ and filtered. The remaining solution was concentrated under a reduced pressure, gaining 46.26 g of a compound 3 (yield=60%).

### (iv) Preparation of 3-(3,4-bisoctyloxyphenyl)-pyridine (Compound 4)

57.55 g of a compound 3 and 19.75 g of 3-bromopyridine were dissolved in 300 ml of a mixed solution of dimethoxyethane (DME) and H₂O, which were mixed in a ratio of 1.5:1, and 0.1 eq of Pd(OAc)₂ and 0.1 eq of tris-o-tolyl phosphine were added thereto. Then, 2.5 eq of K₂CO₃ was dissolved in 150 ml of a mixed solution of DME and H₂O, which were mixed in a ratio of 1.5:1. This solution was added to the former solution in a dropwise fashion. The resulting solution was heated for one hour and refluxed for a reaction.

When the reaction was complete, 300 ml of ethyl acetate and 200 ml of water were added thereto to separate an organic layer. The organic layer was dehydrated by using 30 g of MgSO₄ and then filtered. The remaining solution was concentrated under a reduced pressure.

A solution of n-hexane and ethylacetate mixed in a ratio 10:1 was used as a development solvent to perform a silica gel column chromatography, gaining 35.90 g of a compound 4 (yield=70%).

### (v) Preparation of 3-(2-bromo-4,5-bisoctyloxyphenyl)pyridine (Compound 5)

40.19 g of a compound 4 was dissolved in 350 ml of methylene chloride, and thereafter, cooled down to 0 to 5° C. Then, 1.1 eq of NBS was added to the solution in a smally-divided amount. The resulting mixture was allowed to stand for a reaction at room temperature for 2 hours.

When the reaction was complete, 200 ml of water was added thereto and then, agitated together to separate an organic layer. The organic layer was washed with 100 ml of a saturated NaHCO₃ solution and 100 ml of salt water, and thereafter dehydrated with 30 g of MgSO₄ and filtered. The remaining solution was concentrated under a reduced pressure. When the reaction was complete, a mixed solution of n-hexane and ethylacetate, which were mixed in a ratio of 10:1, was used as a development solvent to perform a silica gel column chromatography, gaining 35.62 g of a compound 5 (yield=83%).

### (vi) Preparation of 9-(4,5-bisoctyloxy-2-pyridine-3-yl-phenyl)-2,7-dibromo-9H-fluorene-9-ol (Compound 6)

35.62 g of a compound 5 was dissolved in 500 ml of anhydrous THF and cooled down to −78° C. 35 ml (1.7 M) of t-BuLi was slowly added to the reaction solution in a dropwise fashion and then, agitated together. Also, a solution prepared by dissolving 10.00 g (0.0296 mol) of 2,7-dibromo-9-fluorene in 300 ml of anhydrous THF was added to the reaction solution in a dropwise fashion for 30 minutes.

When the reaction was complete, the reaction solution was concentrated under a reduced pressure. 200 ml of ethyl and 200 ml of acetate salt-saturated water were added to the residue to separate an organic layer. The organic layer was dehydrated by using 30 g of MgSO₄ and then filtered. The remaining solution was concentrated under a reduced pressure. A mixed solution of n-hexane and ethylacetate, which was prepared in a ratio of 3:1, was used as a development solvent to perform a silica gel column chromatography to the concentrated residue, gaining 25.04 g of a compound 6 (yield=46%).

### (vii) Preparation of 6,7-bis(n-octyloxy)-9H-indeno[2,1-b]pyridine-9-spiro-9′-2,7-dibromo-9H-fluoren (Compound 7)

25.04 g of compound 6 was dissolved in 250 g of polyphosphoric acid and thereafter, was heated up to 100-110° C. Then, the reaction solution was reacted at the same temperature for 2 hours and then, cooled down to a room temperature.

When the reaction was complete, the reaction solution was added to 300 ml of ice water in a dropwise fashion, so that it could be crystallized and thereafter, filtered. The gained solid was dissolved in a mixed solution of 300 ml of CHCl₃ and 300 ml of water and then, regulated to have pH10 to 11 by using a 40%-NaOH solution. Then, an organic layer was separated and then, washed with salt-saturated water. Then, it was dehydrated and decolorized with 20 g of MgSO₄ and 10 g of activated carbon and then, filtered.

The remaining solution was concentrated under a reduced pressure to gain a residue. The residue was recrystallized by using 500 ml of acetone, obtaining 14.5 g of a compound 7 (yield=58%).

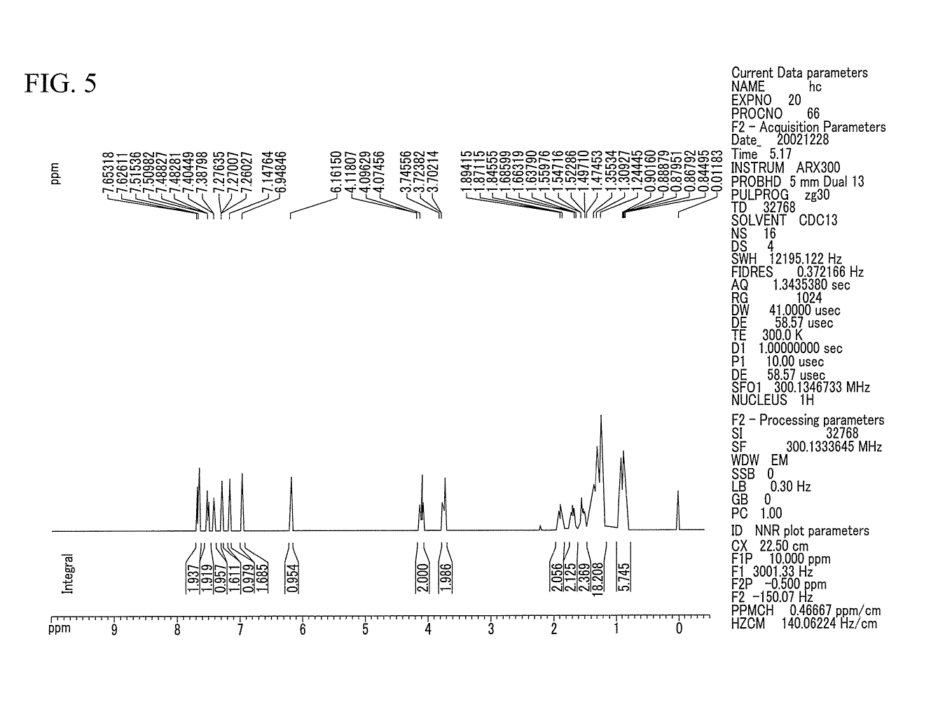

Compound 7 was examined regarding its structure through a ¹H-NMR, and the result was provided in FIG. 4.

### EXAMPLE 2

### Preparation of a Dibromo Compound 11

A dibromo compound 11 was synthesized according to the following Reaction Scheme 2.

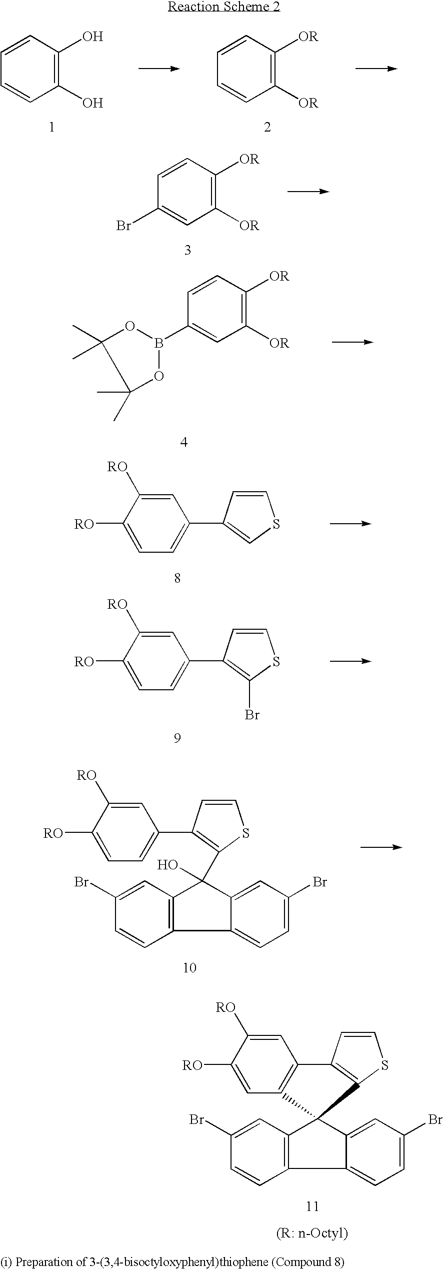

39.13 g of a compound 4 of Example 1 and 39.13 g of 3-bromothiophene were dissolved in 300 ml of a mixed solution of DME and H₂O, which was prepared in a ratio of 1.5:1, and then, 0.1 eq of Pd(OAc)₂ and 0.1 eq of tris-o-tolyl phosphine were added thereto. In addition, 2.5 eq of K₂CO₃ was dissolved in 150 ml of a mixed solution of DME and H₂O, which was prepared in a ratio of 1.5:1. Then, this solution was added to the former solution in dropwise fashion. The mixture solution was heated and refluxed for a reaction for one hour.

When the reaction was complete, 300 ml of ethyl acetate and 200 ml of water were added thereto to separate an organic layer. The organic layer was dehydrated with 30 g of MgSO₄ and then, filtered. The remaining solution was concentrated under a reduced pressure. A mixture of n-hexane and ethylacetate, which was prepared in a ratio of 10:1, was used as a development solvent to perform a silica gel column chromatography, gaining 28.10 g of a compound 8 (yield=79%).

### (ii) Preparation of 3-(3,4-bis-octyloxy-phenyl)-2-bromo-thiophene (Compound 9)

28.10 g of compound 8 was dissolved in 200 ml of acetic acid and 200 ml of CHCl₃, and then cooled down to −78° C. 35 ml (1.7 M) of t-BuLi was slowly added to the reaction solution in a dropwise fashion and then, agitated for an hour. Another solution prepared by dissolving 10.00 g (0.0296 mol) of 2,7-dibromo-9-fluorene in 300 ml of anhydrous THF was added to this reaction solution in a dropwise fashion.

When the reaction was complete, the reaction solution was concentrated under a reduced pressure. The residue was treated with 200 ml of ethyl acetate and 200 ml of salt-saturated water to separate an organic layer. The organic layer was dehydrated with 30 g of MgSO₄ and then, filtered. The remaining solution was concentrated under a reduced pressure again. Then, a mixture of n-hexane and ethylacetate, which was prepared in a ratio of 3:1, was used as a development solvent to perform a silica gel column chromatography, gaining 29.50 g of a compound 9 (yield=88%).

### (iii) Preparation of 9-[3-(3,4-bis-octyloxy-phenyl)-thiophene-2-yl]-2,7-dibromo-9H-fluorene-9-ol (Compound 10)

12.39 g of a compound 9 was dissolved in 250 g of polyphosphoric acid, and thereafter heated up to 100 to 110° C. The reactant mixture was reacted at the same temperature for 2 hours and cooled down to a room temperature.

When the reaction was complete, the reactant mixture was added to 300 ml of ice water in a dropwise fashion, so that it could be crystallized. The produced solid was filtered.

The filtered solid was dissolved in 300 ml of CHCl₃ and 300 ml of water. Then, a NaOH solution was added thereto to regulate a pH level into 10 to 11. Then, an organic layer was separated and washed with salt-saturated water. It was dehydrated and decolorized with 20 g of MgSO₄ and 10 g of activated carbon and thereafter, filtered.

The remaining solution was concentrated under a reduced pressure. Then, a mixture of n-hexane and ethyl acetate, which was prepared in a ratio of 15:1, was used as a development solvent to perform a silica gel column chromatography, gaining 12.45 g of a compound 10 (yield=66%).

### (iv) Preparation of 5,6-bis(n-octyloxy)-8H-indeno[2,1-b]thiophene-9-spiro-9′-2,7-dibromo-9H-fluorene (Compound 11)

12.45 g of a compound 10 was used in the same method as a compound 7 of Example 1 was prepared. The other reagents were used in the same equivalent number. When the reaction was complete, 200 ml of MeOH was added to the reaction solution for crystallization and then, filtered. Then, 400 ml of acetone was added thereto to perform recrystallization regarding it, gaining 10.57 g of a compound 11 (yield=87%).

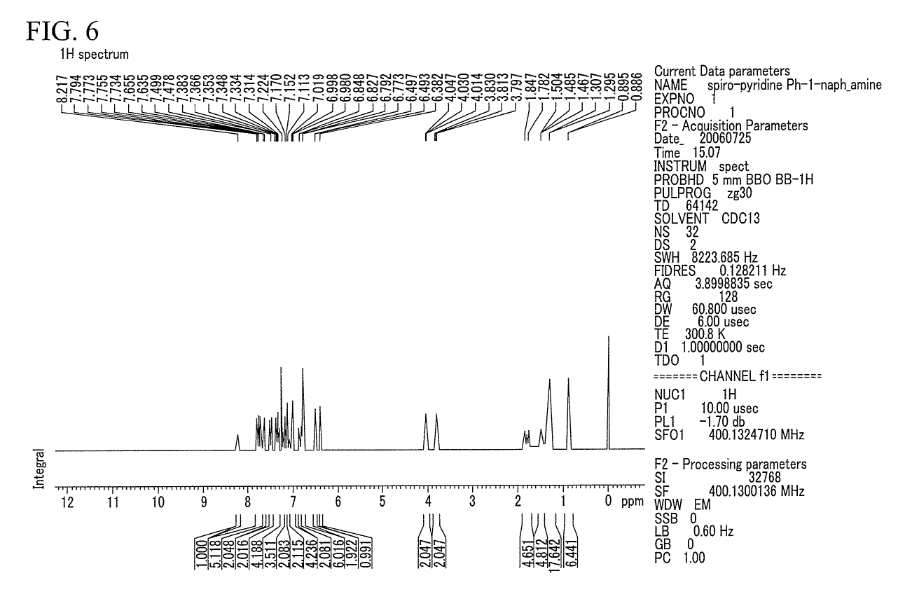

Compound 11 was examined regarding its structure through a ¹H-NMR. The result is provided in FIG. 5.

### EXAMPLES 3 TO 7

### Preparation of Compounds 12 to 16

Compounds 12 to 16 were respectively prepared according to the following Reaction Scheme 3.

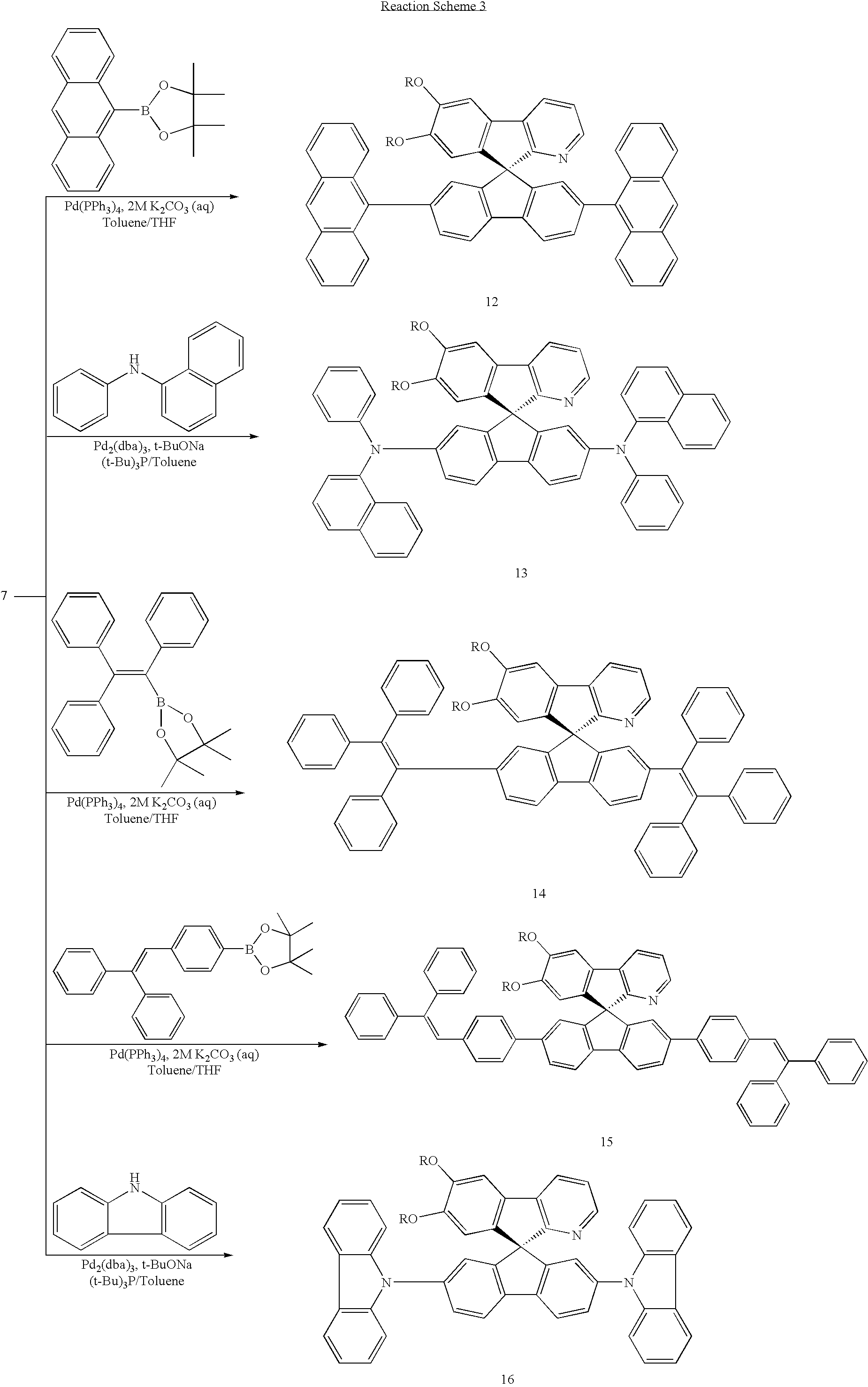

### (i) EXAMPLES 3, 5, AND 6

### Preparation of Compounds 12, 14, and 15

Compound 7 of Example 1 was used in a Pd 0-mediated Suzuki Aryl Coupling method according to the Reaction Scheme 3 to synthesize compounds 5 12, 14, and 15.

For example, a compound 12 was synthesized as follows.

4.67 g (6.38 mmol) of a compound 7, 4.08 g (13.4 mmol, 2.1 eq) of 2-(anthracene-9-yl)-4,4,5,5-tetramethyl-1,3,2-dioxaborolane as an anthracene Borate derivative, and 1 mol % (73.7 mg) of Pd(PPh₃)₄ were dissolved in 30 mL of anhydrous toluene and 30 mL of THF, and thereafter, 16 mL (5 eq) of 2MK₂CO₃ was added thereto. Then, the mixture solution was reacted at 100° C. for 36 hours. When the reaction was complete, the reaction solution was extracted with water and ethylacetate. Then, the extract was dried and thereafter, recrystallized with diethyl ether and chloroform, gaining 4.91 g of a product (yield=83%). The produced compound 12 was examined regarding its structure through a ¹H-NMR.

Compound 12 of Example 3 had 925.49 of m/z. The CHN element analysis of C₆₈H₆₃NO₂ was as follows: C=88.18; H=6.86; N=1.51, and its experiment analysis was as follows: C=88.16; H=6.88; N=1.52.

Compound 14 of Example 5 had 1081.58 of m/z. The CHN element analysis of C₈₀H₇₅NO₂ was as follows: C=88.77; H=6.98; N=1.29, and its experiment analysis was as follows: C=88.77; H=6.99; N=1.30.

Compound 15 of Example 6 had 1081.58 of m/z. The CHN element analysis of C₈₀H₇₅NO₂was as follows C=88.77; H=6.98; N=1.29, and its experiment analysis was as follows: C=88.75; H=6.98; N=1.28.

### (ii) EXAMPLES 4 AND 7

### Preparation of Compounds 13 and 16

Compounds 13 and 16 were synthesized from a compound 7 of Example 1 according to Reaction Scheme 3 in a Pd(O)-mediated C—N Aryl Coupling method.

For example, a compound 13 was synthesized as follows.

1.17 g (1.6 mmol) of a compound 7, 0.724 g (3.3 mmol) of N-phenyl-1-naphthylamine, 0.059 g (0.64×10⁻⁴ mol) of Pd₂(dba)₃, 0.465 g (4.835 mmol) of t-BuONa, and 0.016 g (0.81×10⁻⁴ mol) of (t-Bu)₃P were all put in a 100 mL round flask under a nitrogen atmosphere, and then, 40 mL of anhydrous toluene was added thereto. Its temperature was slowly increased up to 110° C. by using an oil bath while agitating it.

Then, the reactant mixture was reacted for 48 hours. When the reaction was complete, the organic layer was treated with water and CHCl₃ and thereafter, washed with 500 mL of an 1 N hydrochloric acid aqueous solution. After the organic solvent was all removed under a reduced pressure, a gained solid was purified by using a Train sublimation device.

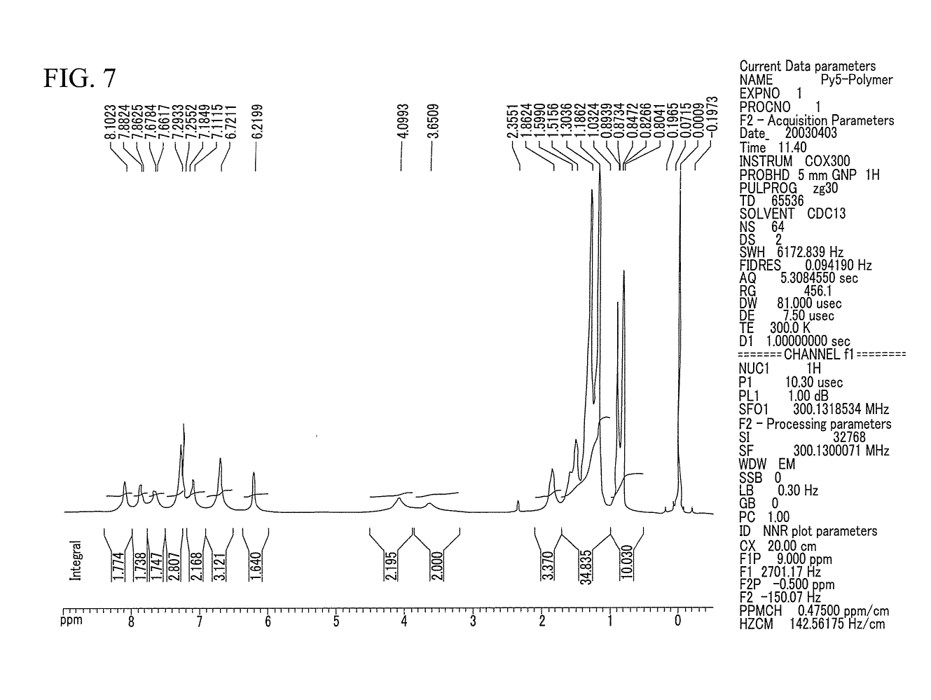

About 1.53 g of a compound 13 was gained (yield=95%) and examined regarding its structure through a ¹H-NMR. The ¹H-NMR spectrum of compound 13 was provided in FIG. 6.

Compound 13 of Example 4 had 1007.54 of m/z. The CHN element analysis of C₇₂H₆₉N₃O₂ was C=85.76; H=6.90; N=4.17, and its experiment analysis was C=85.79; H=6.93; N=4.16.

Compound 16 of Example 7 had 903.48 of m/z. The CHN element analysis of C₆₄H₆₁N₃O₂ was C=85.01; H=6.80; N=4.65, and its experiment analysis was C=85.02; H=6.82; N=4.64.

### EXAMPLES 8 TO 12

### Preparation of Compounds 17 to 21

Compounds 17 to 21 were respectively prepared according to the following Reaction Scheme 4.

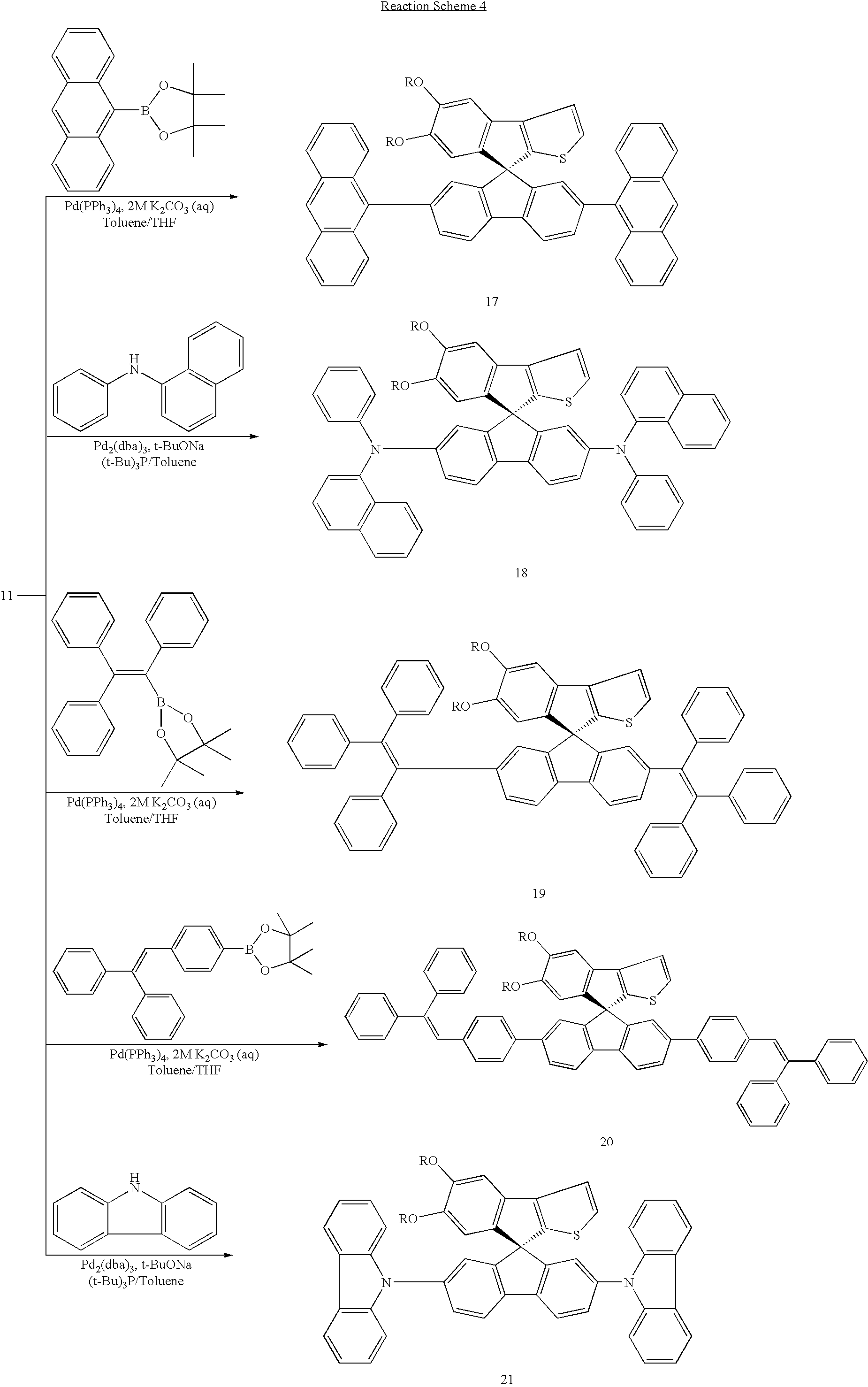

### (i) EXAMPLES 8, 10 AND 11

### Preparation of Compounds 17, 19, and 20

Compounds 17, 19, and 20 were synthesized from a compound 11 of Example 2 according to Reaction Scheme 4 in a Pd(0)-mediated Suzuki Aryl Coupling method.

For example, a compound 17 was synthesized as follows.

4.70 g (6.38 mmol) of a compound 11, 4.08 g (2.1 eq, 13.4 mmol) of 2-(anthracene-9-yl)-4,4,5,5-tetramethyl-1,3,2-dioxaborolane as an anthracene Borate derivative, and 73.7 mg (1 mol %) of Pd(PPh₃)₄ were dissolved in 30 mL of anhydrous toluene and 30 mL of THF, and then, 16 mL (5 eq) of 2M K₂CO₃ was added thereto. The mixture was reacted at 100° C. for 36 hours.

When the reaction was complete, the reactant solution was extracted with water and ethylacetate. Then, the extract was dried and recrystallized with diethyl ether and chloroform, gaining 5.20 g of a product (yield=88%). The produced compound 17 was examined regarding its structure through a ¹H-NMR.

Compound 17 of Example 8 had 930.45 of m/z. The element analysis of C₆₇H₆₂O₂S was C=86.41; H=6.71, and its experiment analysis was C=86.43; H=6.72,

Compound 19 of Example 10 had 1086.54 of m/z. The element analysis of C₇₉H₇₄O₂S was C=87.25; H=6.86, and its experiment analysis was C=87.26; H=6.87.

Compound 20 of Example 11 had 1086.54 of m/z. The element analysis of C₇₉H₇₄O₂S was C=87.25; H=6.86, and its experiment analysis was C=87.23; H=6.89.

(ii) EXAMPLES 9 AND 12

### Preparation of Compounds 18 and 21

Compounds 18 and 21 were synthesized from a compound 11 of Example 2 according to Reaction Scheme 4 in a Pd(0)-mediated C—N Aryl Coupling method.

For example, a compound 18 was synthesized as follows.

1.18 g (1.6 mmol) of a compound 11, 0.724 g (3.3 mmol) of N-phenyl-1-naphthylamine, 0.059 g (0.64×10⁻⁴ mol) of Pd₂(dba)₃, 0.465 g (4.835 mmol) of t-BuONa, and 0.016 g (0.81×10⁻⁴ mol) of (t-Bu)₃P were all put in a 100 mL round flask under a nitrogen atmosphere, and then, 40 mL of anhydrous toluene was added thereto.

Then, its temperature was slowly increased up to 110° C. by using an oil bath while agitating it. The reactant mixture was reacted for 48 hours. When the reaction was complete, the mixture was worked up with water and CHCl₃. Then, an organic layer was washed with 500 mL of an 1 N hydrochloric acid aqueous solution. Then, an organic solvent was all removed under a reduced pressure, gaining a solid. The solid was purified by using a Train sublimation device.

1.50 g of a compound 18 was gained (yield=93%). Compound 18 was examined regarding its structure through a ¹H-NMR.

Compound 18 of Example 9 had 1012.50 of m/z. The CHN element analysis of C₇₁H₆₈N₂O₂S was C=84.15; H=6.76; N=2.76, and its experiment analysis was C=84.16; H=6.79; N=2.77.

Compound 21 of Example 12 had 908.44 of m/z. The CHN element analysis of C₆₃H₆₀N₂O₂S was C=83.22; H=6.65; N=3.08, and its experiment analysis was C=83.20; H=6.63; N=3.06.

### EXAMPLES 13 AND 14

### Preparation of Compounds 22 and 23

Compounds 7 and 11 prepared in Examples 1 and 2 were treated with 1 equivalent of n-butyl lithium to substitute lithium for bromine atoms at one side, and thereafter, prepared into compounds 22 and 23 according to the following Reaction Scheme 5 through a coupling reaction using Fe(acac)₃.

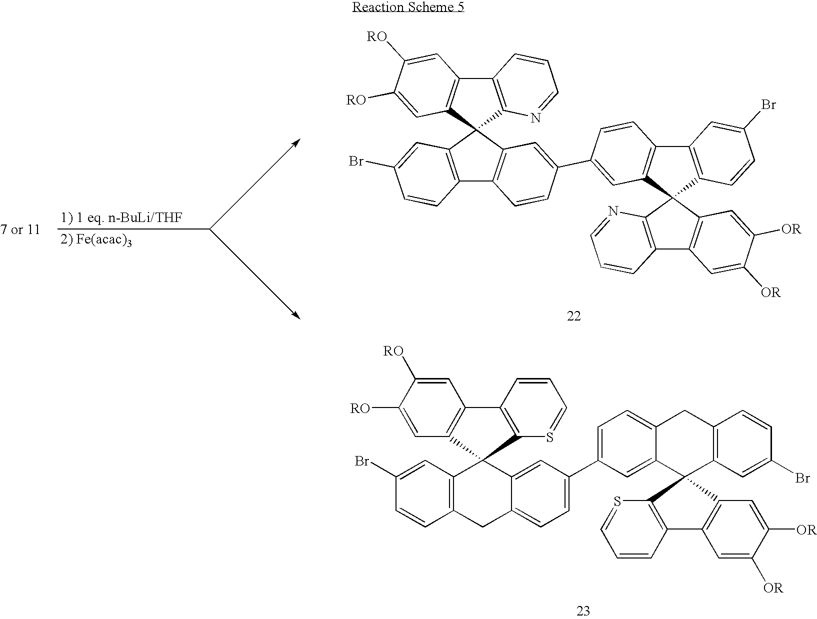

The yield rate of compounds 22 and 23 was respectively about 60% and 72%. Their structure was examined through an NMR.

Compound 22 of Example 13 had 1302.52 of m/z. The element analysis of C₈₀H₉₀Br₂N₂O₄ was C=73.72; H=6.96, and its experiment analysis was C=73.70; H=6.71.

Compound 23 of Example 14 had 1312.45 of m/z. The element analysis of C₇₈H₈₈Br₂O₄S₂ was C=71.33; H=6.75, and its experiment analysis was C=71.30; H=6.79.

### EXAMPLES 15 TO 19

### Preparation of Compounds 24 to 28

Compounds 24 to 28 were respectively prepared according to the following Reaction Scheme 6 in the same method as in Example 3 except that a compound 22 of Example 13 was used instead of a compound 7.

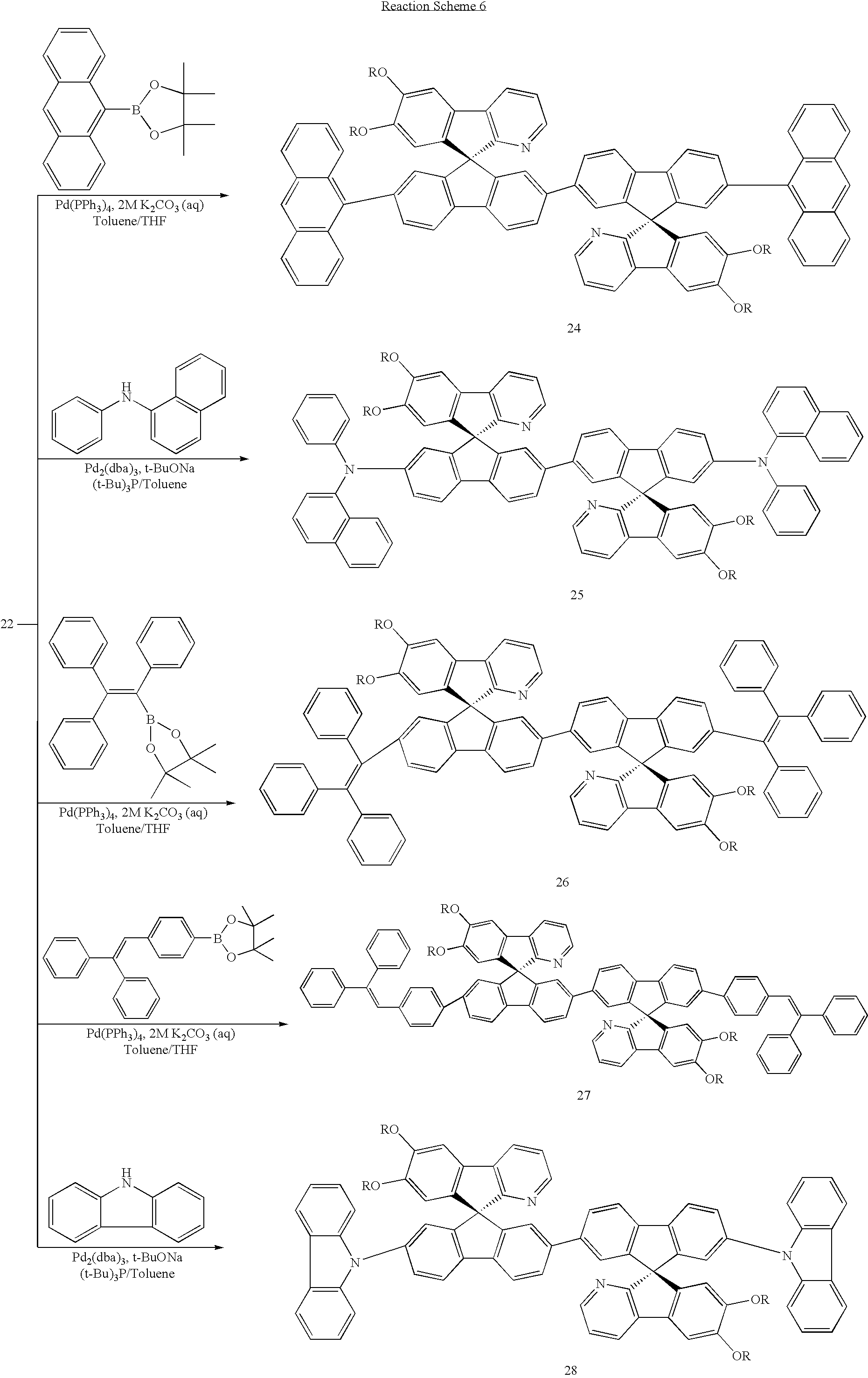

The compounds 24 to 28 were examined regarding their structure through an NMR.

Compound 24 of Example 15 had 1043.29 of m/z. The element analysis of C₇₆H₃₉N₂O₄ was C=87.42; H=3.76; N=2.68, and its experiment analysis was C=87.43; H=3.80; N=2.65.

Compound 25 of Example 16 had 1579.89 of m/z. The element analysis of C₁₁₂H₁₁₄N₄O₄ was C=85.13; H=7.27; N=3.55, and its experiment analysis was C=85.10; H=7.23; N=3.54.

Compound 26 of Example 17 had 1653.93 of m/z. The element analysis of C₁₂₀H₁₂₀N₂O₄ was C=87.13; H=7.31; N=1.69, and its experiment analysis was C=87.10; H=7.39; N=1.62.

Compound 27 of Example 18 had 1653.93 of m/z. The element analysis of C₁₂₀H₁₂₀N₂O₄was C=87.13; H=7.31; N=1.69, and its experiment analysis was C=87.14; H=7.32; N=1.68.

Compound 28 of Example 19 had 1475.82 of m/z. The element analysis of C₁₀₄H₁₀₆N₄O₄ was C=84.63; H=7.24; N=3.80, and its experiment analysis was C=84.62; H=7.25; N=3.81.

### EXAMPLES 20 TO 24

### Preparation of Compounds 29 to 33

Compounds 29 to 33 were prepared according to the following Reaction Scheme 7 in the same method as in Example 8 except that a compound 23 of Example 14 instead of a compound 11 was used.

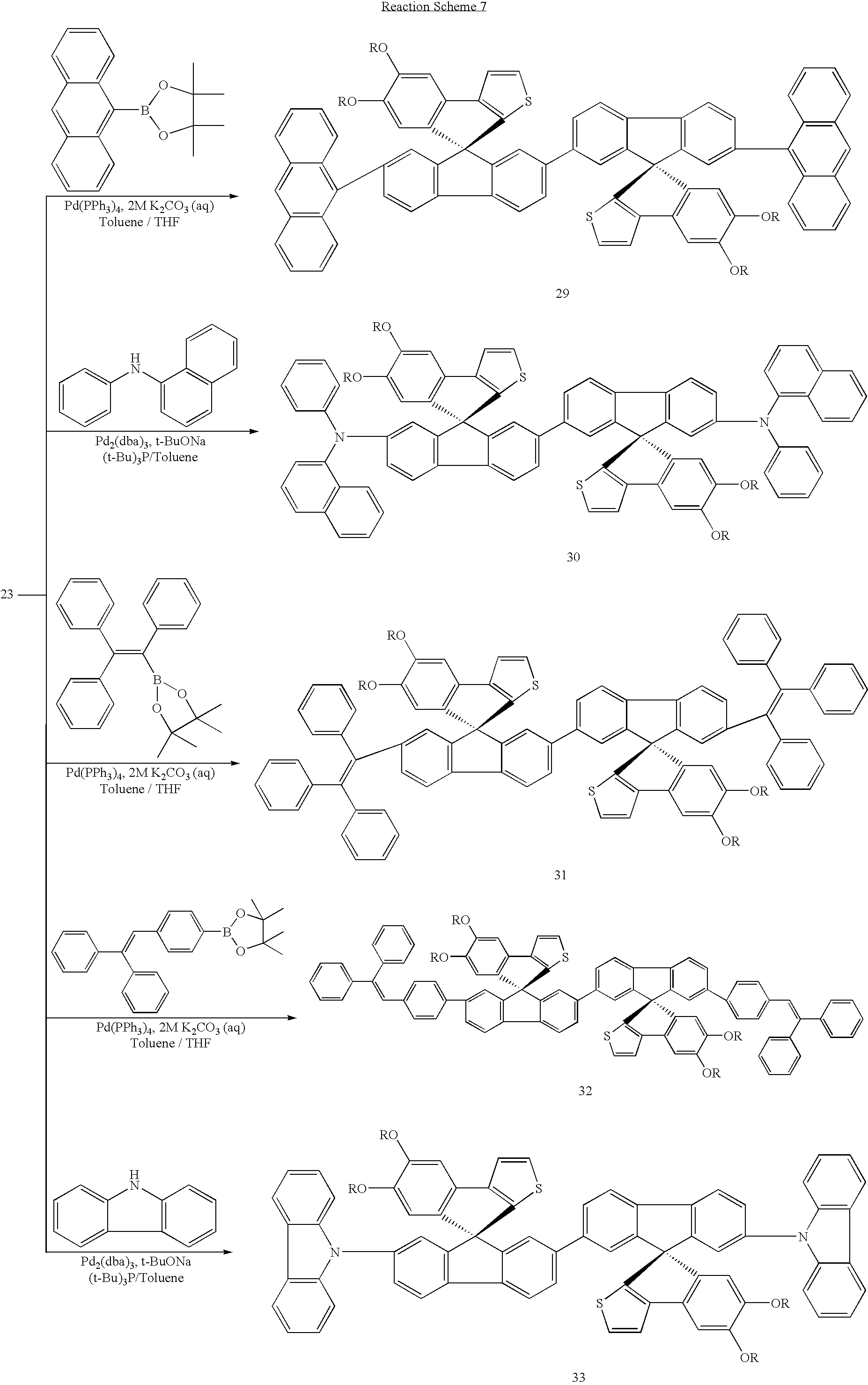

The compounds 29 to 33 were examined about their structure through an NMR.

Compound 29 of Example 20 had 1507.76 of m/z. The element analysis of C₁₀₆H₁₀₆O₄S₂ was C=84.42; H=7.08, and its experiment analysis was C=84.40; H=7.12.

Compound 30 of Example 21 had 1589.81 of m/z. The element analysis of C₁₁₀H₁₁₂N₂O₄S₂ was C=83.08; H=7.10; N=1.76, and its experiment analysis was C=83.09; H=7.14; N=1.75.

Compound 31 of Example 22 had 1663.85 of m/z. The element analysis of C₁₁₈H₁₁₈O₄S₂was C=85.16; H=7.15, and its experiment analysis was C=85.17; H=7.16.

Compound 32 of Example 23 had 1663.85 of m/z. The element analysis of C₁₁₈H₁₁₈O₄S₂ was C=85.16; H=7.15, and its experiment analysis was C=85.12; H=7.15.

Compound 33 of Example 24 had 1485.75 of m/z. The element analysis of C₁₀₂H₁₀₄N₂O₄S₂ was C=82.44; H=7.05; N=1.89, and its experiment analysis was C=82.46; H=7.07; N=1.88.

### EXAMPLE 25

### Synthesis of the Polymer 1

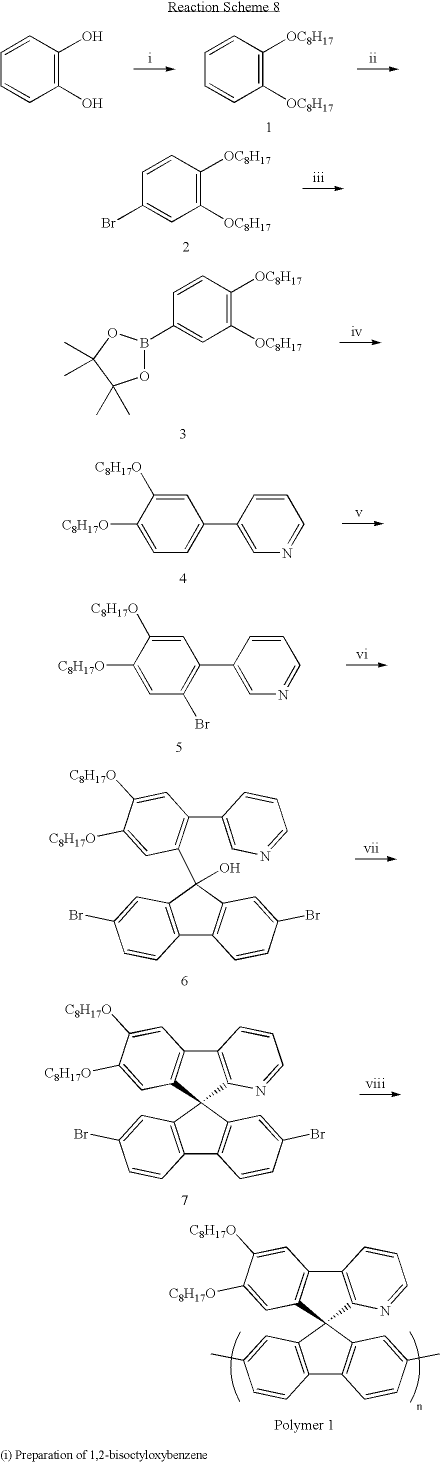

19 g of catechol was dissolved in 200 ml of acetonitrile, and then, 2.5 eq of 1-bromooctane, 2.5 eq of K₂CO₃, and 0.1 eq of Kl were added thereto. The mixture was heated and refluxed for 24 hours. When the reaction was complete, the resulting mixture was filtered. Then, the obtained organic layer was concentrated under reduced pressure. Next, the residue was dissolved in 200 ml of ethylether, and then washed with 100 ml of water and salt-saturated water to separate the organic layer. The separated organic layer was dehydrated with 20 g of MgSO₄. The remaining solution was concentrated under a reduced pressure, obtaining 57.17 g of a white solid compound (yield=99%). The produced compound was examined regarding the structure through a ¹H-NMR.

### (ii) Preparation of 4-bromo-1,2-bis-octyloxybenzene (Compound 2 in Reaction Scheme 8)

57.17 g of the compound 1 was dissolved in 400 ml of methylene chloride. In addition, 1.1 eq of NBS was dissolved in 100 ml of DMF at 0° C. The latter solution was added to the former one in a dropwise fashion, and thereafter, its temperature was increased and reacted for 2 hours. When the reaction was complete, the reaction solution was twice washed with 200 ml of water. Then, the organic layer was washed with a Na₂S₂O₃. 5H₂O solution, a NaHCO₃ saturated solution, and brine in order, and thereafter treated with MgSO₄ and filtered. Then, the solvent was concentrated under a reduced pressure, gaining 69.01 g of a compound (yield=98%). The produced compound was examined regarding its structure through a ¹H-NMR.

### (iii) 1-(3,4-bisoctyloxyphenyl)-3,3,4,4-tetramethyl-borolane (Compound 3 in Reaction Scheme 8)

69.03 g of compound 2 was put in a 2 L flask, dissolved in 500 ml of anhydrous THF, and then, 1.2 eq of n-BuLi was slowly added thereto at 78° C. in a dropwise fashion. Then, the mixture was agitated for 10 minutes, and thereafter, 1.1 eq of 2-isoproxy-4,4,5,5-tetramethyl-1,3,2-dioxaborolane was added thereto at the same temperature in a dropwise fashion. The resulting mixture was agitated for one hour. When the reaction was complete, 300 ml of ethyl acetate and 300 ml of water were added to the reaction solution to separate an organic layer. The organic layer was washed with 150 ml of a saturated NaHCO₃ solution and 150 ml of salt water, and thereafter treated with MgSO₄. Then, it was filtered, and the remaining solution was concentrated under a reduced pressure to gain 46.26 g of a desired compound (yield=60%).

### (iv) 3-(3,4-bisoctyloxyphenyl)-pyridine (Compound 4 in Reaction Scheme 8)

57.55 g of the compound 3 and 19.75 g of 3-bromopyridine were dissolved in 300 ml of DME and H₂O mixed in a ratio of 1.5:1, and 0.1 eq Pd(OAc)₂ and 0.1 eq of tris-o-tolyl phosphine were added thereto. In addition, 2.5 eq of K₂CO₃ was dissolved in 150 ml of DME and H₂O mixed in a ratio of 1.5:1. The solution was added in a dropwise fashion to the above mixture. The resulting mixture was heated and refluxed for one hour for a reaction. When the reaction was complete, 300 ml of ethyl acetate and 200 ml of water were added to the reaction solution to separate an organic layer. The organic layer was dehydrated with 30 g of MgSO₄, and then filtered. The remaining solution was concentrated under a reduced pressure. Then, a silica gel column was performed to the resulting solution by using a solution of n-hexane and ethylacetate mixed in a ratio of 10:1 as a development solvent, gaining 35.90 g of a compound (yield=70%).

### (v) 3-(2-bromo-4,5-bisoctyloxyphenyl)pyridine (Compound 5 in Reaction Scheme 8)

40.19 g of the compound 4 was dissolved in 350 ml of methylene chloride, and then cooled down to 0 to 5° C. On the other hand, 1.1 eq of NBS was minutely ground, and thereafter added to the reaction solution. Then, it was allowed to stand for a reaction at room temperature for 2 hours. When the reaction was complete, 200 ml of water was added to the reaction solution. Then, the mixture solution was agitated, and thereafter, an organic layer was separated. The separated organic layer was washed with 100 ml of a saturated NaHCO₃ solution and 100 ml of salt water, thereafter dehydrated with 30 g of MgSO₄, and filtered. The remaining solution was concentrated under a reduced pressure. When the reaction was complete, a silica gel column was performed by using a solution of n-hexane and ethylacetate mixed in a ratio of 10:1 as a development solvent, gaining 35.62 g of a compound (yield=83%).

### (vi) 9-(4,5-bisoctyloxy-2-pyridine-3-yl-phenyl)-2,7-dibromo-9H-fluorene-9-al (Compound 6 in Reaction Scheme 8)

35.62 g of the compound 5 was dissolved in 500 ml of anhydrous THF, and then cooled down to −78° C. 35 ml (1.7 M) of t-BuLi was slowly added to the reaction in a dropwise fashion and agitated for one hour. In addition, 10.00 g (0.0296 mol) of 2,7-dibromo-9-fluorene was dissolved in 300 ml of anhydrous THF. The solution was added to the above reaction solution for 30 minutes in a dropwise fashion. When the reaction was complete, the reaction solution was concentrated under a reduced pressure. The residue was treated with 200 ml of ethyl acetate and 200 ml of salt-saturated water to separate an organic layer. The organic layer was dehydrated with 30 g of MgSO₄, then filtered and concentrated under a reduced pressure again. Then, a silica gel column was performed regarding the gained residue by using a solution of n-hexane and ethylacetate mixed in a ratio of 3:1 as a development solvent, gaining 25.04 g of a compound (yield=46%).

### (vii) 6,7-bis(n-octyloxy)-9H-indeno[2,1-b]pyridine-9-spiro-9′-2,7-dibromo-9H-fluorene (Compound 7 in Reaction Scheme 8)

25.04 g of a compound 6 was dissolved in 250 g of polyphosphoric acid, and then heated up to 100 to 110° C. The solution was reacted at the same temperature for 2 hours. Then, the reaction mixture was cooled down to room temperature. When the reaction was complete, the reaction mixture was added to 300 ml of ice water in a dropwise fashion for crystallization. The produced solid was filtered. The gained solid was dissolved in 300 ml of CHCl₃ and 300 ml of water and thereafter, its pH was regulated to be in a range of 10 to 11 with a 40% NaOH solution. Then, an organic layer was separated. The separated organic layer was washed with salt-saturated water, dehydrated and decolored by adding 20 g of MgSO₄ and 10 g of activated carbon, and then filtered. The remaining solution was concentrated under a reduced pressure. The gained residue was recrystalyzed by using 500 ml of acetone, obtaining 14.5 g of a compound (yield=58%). The produced compound was examined regarding its structure through a ¹H-NMR. The result was provided in FIG. 2.

### (viii) Preparation of the Polymer 1

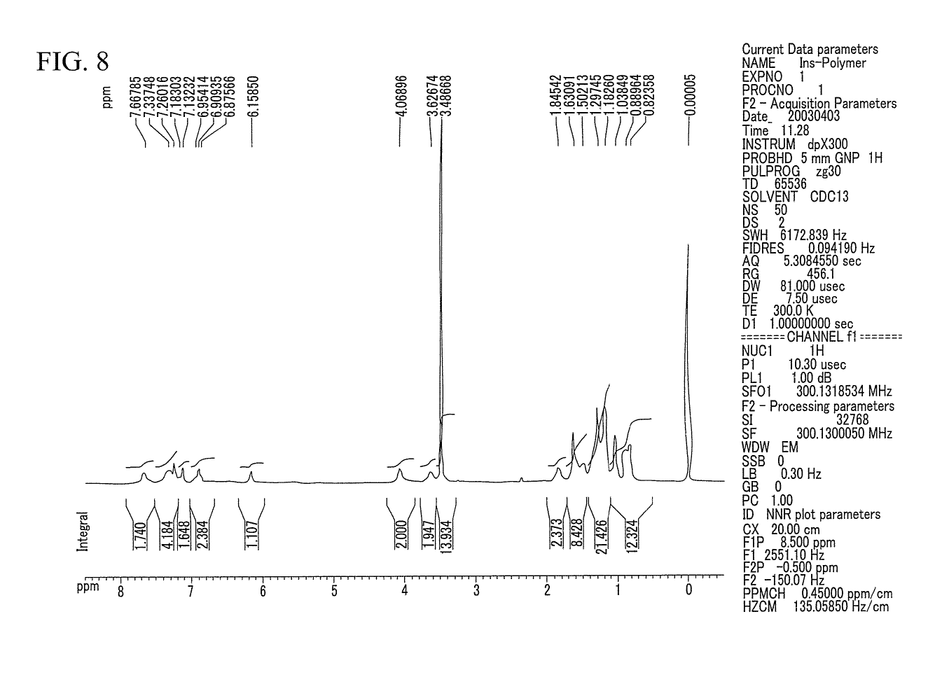

A Schlenk flask was internally several times vacuumed and refluxed with nitrogen to completely remove moisture therein. Then, 880 mg of Ni(COD)₂ (3.2 mmol) and 500 mg of bipyridal (3.2 mmol) were put in the flask inside a glove box, and then several times vacuumed and refluxed with nitrogen again. Next, 10 ml of anhydrous DMF, 34 6 mg (3.2 mmol) of COD, and 10 ml of anhydrous toluene were added thereto under a nitrogen current. The resulting mixture was agitated at 80° C. for 30 minutes, and 1.60 mmol of compound 7 was diluted, and then added to 10 ml of toluene. After 10 ml of toluene was added to wash all the materials on the wall of the flask, the mixture was agitated at 80° C. for 4 days. Four days later, 1 ml of bromo pentafluorobenzene was added to the resulting mixture, and then agitated at 80° C. for about a day again. When the agitation was complete, the resulting mixture was cooled down to 60° C. The reaction mixture was poured into a solution of HCl, acetone, and methanol mixed in a ratio of 1:1:2 to produce precipitations, and thereafter, agitated for more than 12 hours. The precipitations were obtained by using a gravity filter and then dissolved in a small amount of chloroform. The solution was reprecipitated in methanol. The precipitations were obtained with a gravity filter, and thereafter, a soxhlet was sequentially performed by using methanol and chloroform. The obtained chloroform solution was appropriately concentrated and thereafter, reprecipitated in methanol, obtaining a final product, that is, a polymer 1 (yield: 85%). The produced compound was identified through a ¹H-NMR. The result was provided in FIG. 7. The prepared polymer respectively had a number average molecular weight of 79,000 and a weight average molecular weight of 156,000.

### EXAMPLE 26

### Preparation of a Polymer 2

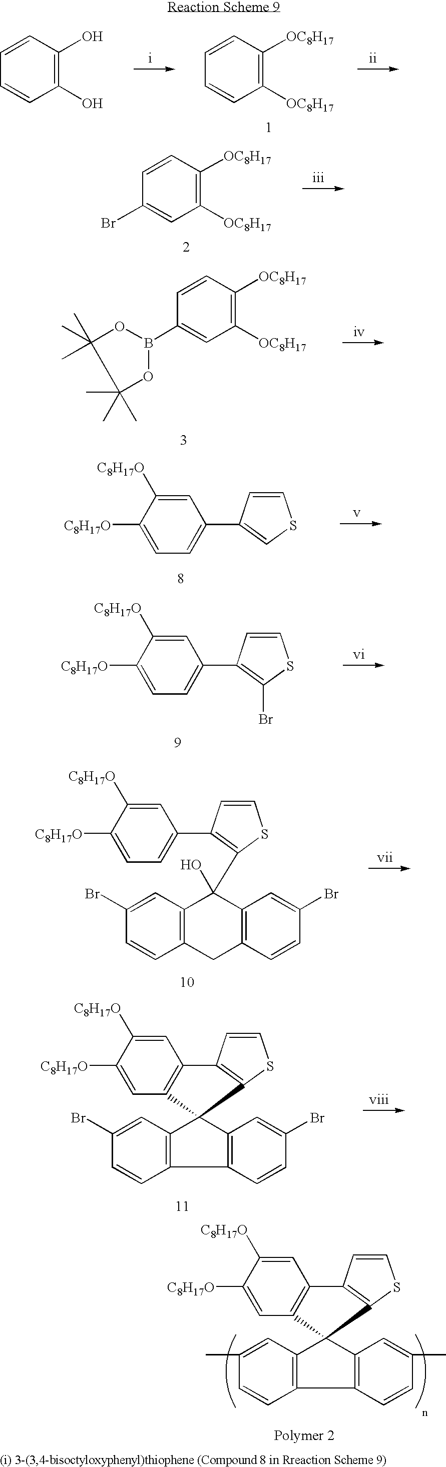

39.13 g of a compound 3 according to Example 25 and 13.86 g of 3-bromopyridine were dissolved in 300 ml of a DME and H₂O solution mixed in a ration of 1.5:1, and 0.1 eq of Pd(OAc)₂ and 0.1 eq of tris-o-tolyl phosphine were added thereto. On the other hand, 2.5 eq of K₂CO₃was dissolved in 150 ml of a DME and H₂O solution mixed in a ratio of 1.5:1. This solution was added to the former solution in a dropwise fashion, and then heated and refluxed for an hour. When the reaction was complete, 300 ml of ethyl acetate and 200 ml of water were added to the reaction solution to separate an organic layer. The organic layer was dehydrated with 30 g Of MgSO₄, filtered, and concentrated under reduced pressure. Then, a silica gel column was performed by using a solution of n-hexane and ethylacetate mixed in a ratio of 10:1 as a development solvent, obtaining 28.10 g of a compound (yield=79%).

### (ii) 3-(3,4-Bis-octyloxy-phenyl)-2-bromo-thiophene (Compound 9 in Reaction Scheme 9)

28.01 g of a compound 8 was dissolved in 200 ml of acetic acid and 200 ml of CHCl₃, and thereafter cooled down to −78° C. 35 ml (1.7 M) of t-BuLi was slowly added in a dropwise fashion to the reaction solution, and thereafter agitated for 1 hour. In addition, 10.00 g (0.0296 mol) of 2,7-dibromo-9-fluorene was dissolved in 300 ml of anhydrous THF. The solution was added to the reaction solution for 30 minutes in a dropwise fashion. When the reaction was complete, the reaction solution was concentrated under a reduced pressure. The residue was treated with 200 ml of ethyl acetate and 200 ml of salt-saturated water to separate an organic layer. The organic layer was dehydrated with 30 g of MgSO₄, filtered, and concentrated under a reduced pressure. Then, a silica gel column was performed regarding the concentrated residue by using a solution of n-hexane and ethylacetate mixed in a ratio of 3:1 as a development solvent, gaining 29.50 g of a compound (yield=88%).

### (iii) 9-[3-(3,4-Bis-octyloxy-phenyl)-thiophen-2-yl]-2,7-dibromo-9H-fluoren-9-ol (Compound 10 in Reaction Scheme 9)

12.39 g of a compound 9 was dissolved in 250 g of polyphosphoric, and thereafter heated up to 100 to 110° C. The solution was reacted for 2 hours at the same temperature and cooled down to a normal temperature. When the reaction was complete, the reaction mixture was added to 300 ml of ice water in a dropwise fashion for crystallization. The crystalyzed solid was filtered. The filtered solid was dissolved in 300 ml of CHCl₃ and 300 ml of water, and its pH level was regulated to be in a range of 10 to 11 with a 40% NaOH solution. The organic layer was separated and washed with salt-saturated water, dehydrateand and decolored with 20 g of MgSO₄ and 10 g of activated carbon, and filtered. Then, a silica gel column was performed regarding the residue by using a solution of n-hexane and ethyl acetate mixed in a ratio of 15:1 as a development solvent, gaining 12.45 g of a product (yield=66%).

### (iV) 5,6-bis(n-octyloxy)-8H-indeno[2,1-b]thiophene-9-spiro-9′-2,7-dibromo-9H-fluorene (Compound 11 in Reaction Scheme 9)

12.45 g of a compound 10 and the same number of an equivalent of the other specimens were reacted in the same method as compound 7 of Example 25 was prepared. When the reaction was complete, the reaction solution was treated with 200 ml of MeOH for crystallization. The produced solid was filtered, and then, recrystalized by using 400 ml of acetone, obtaining 10.57 g of a desired compound (yield=87%). The produced compound was identified through a ¹H-NMR. The result is provided in FIG. 4.

### (v) Preparation of the Polymer 2

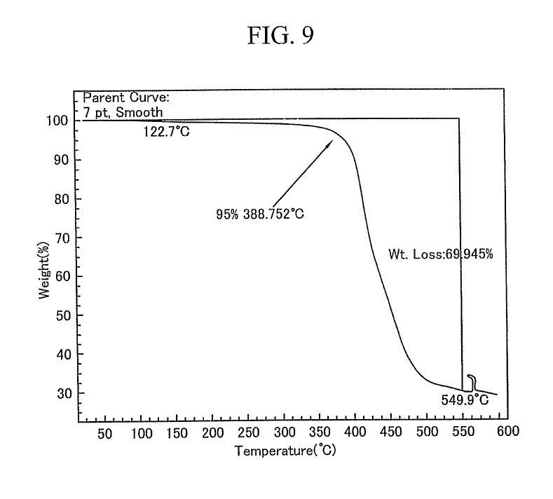

The polymer 2 was synthesized in the same method as the polymer 1 of Example 25 was prepared. The produced polymer 2 was identified through a ¹H-NMR. The result was provided in FIG. 8. The polymer respectively had 67,000 of a number average molecular weight and 134,000 of weight average molecular weight.

### EXAMPLE 27

### Preparation of a Polymer 3

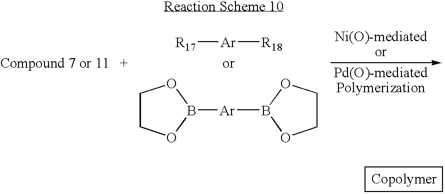

A compound 7 or 11 prepared according to Examples 25 and 26 could be reacted with comonomers such as various dihaloaromatices, aromatic diborate, and the like, to produe a copolymer (refer to a reaction scheme 10). The following reaction scheme 11 shows a representative copolymerization, which is a manufacturing process of a polymer 3.

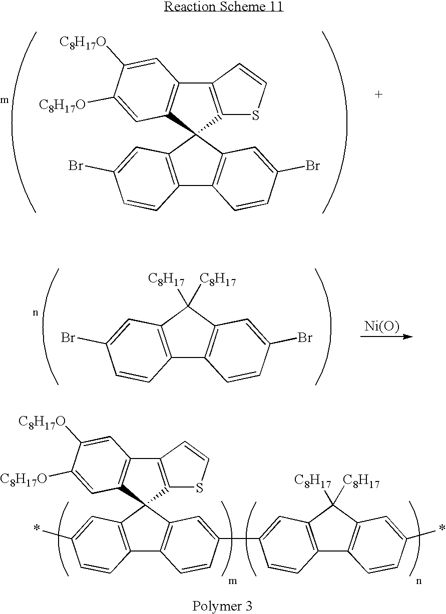

A Schlenk flask was internally vacuumized and refluxed with nitrogen several times to completely remove moisture. 880 mg (3.2 mmol) of Ni(COD)2 and 500 mg (3.2 mmol) of bipyridal were put into the flask in a glove box, and thereafter, the flask was several times vacuumed and refluxed with nitrogen again. Next, 10 ml of anhydrous toluene was added to 10 ml of anhydrous DMF and 346 mg (3.2 mmol) of COD under a nitrogen current. The nitrogen solution was agitated at 80° C. for 30 minutes. On the other hand, 1.52 mmol of a compound 11 and 834 mg (1.52 mmol) of 2,6-dibromo-9,9′- dioctylfluorene were diluted in 10 ml of toluene, and thereafter, added to the agitated nitrogen solution. After 10 ml of toluene was added to wash away all the materials on the flask wall, the mixture solution was agitated at 80° C. for 4 days. 4 days later, 1 ml of bromo pentafluorobenzene was added thereto and agitated at 80° C. for about a day. When the agitation was complete, the resulting solution was cooled down to 60° C. Then, the reaction mixture was poured into a solution of HCl, acetone, and methanol mixed in a ratio of 1:1:2 for precipitation, and thereafter, agitated for more than 12 hours. The precipitations were obtained with a gravity filter. The obtained precipitations were dissolved in a small amount of cloroform. The solution was reprecipitated in methanol. The precipitations were obtained with a gravity filter again. Then, a soxhlet was performed to gain the precipitations by sequentially using methanol and chloroform. The resulting chloroform solution was appropriately concentrated, and thereafter, reprecipitated in methanol, gaining a final product, a polymer 3. The produced compound was identified through a ¹H-NMR. The prepared polymer had respectively a number average molecular weight of 89,000 and a weight average molecular weight of 204,000.

Evaluation of Thermal Characteristics of Prepared Spiro-Compounds

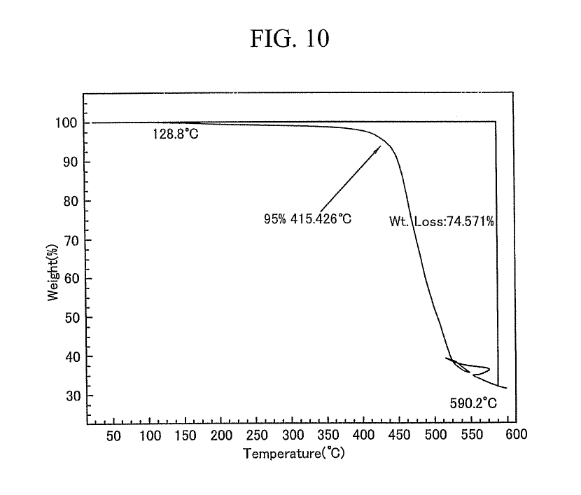

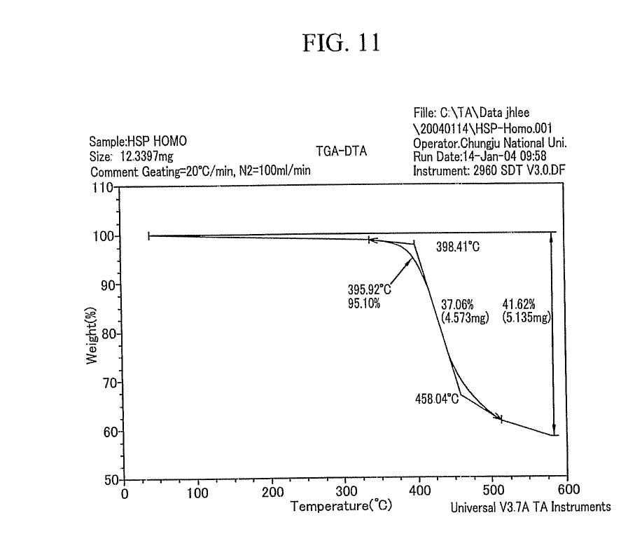

TGA and DSC analyses were performed regarding compound 12 of Example 3 and compound 13 of Example 4. The TGA result of compound 12 is provided in FIG. 9, and that of compound 13 in FIG. 10.

As shown in FIGS. 9 and 10, spiro-compounds 12 and 13 of the present invention turned out to be respectively stable nearly up to 389° C. and 415° C.

As for the DSC analysis, the compound 12 was observed to have a melting point at 196.5° C.

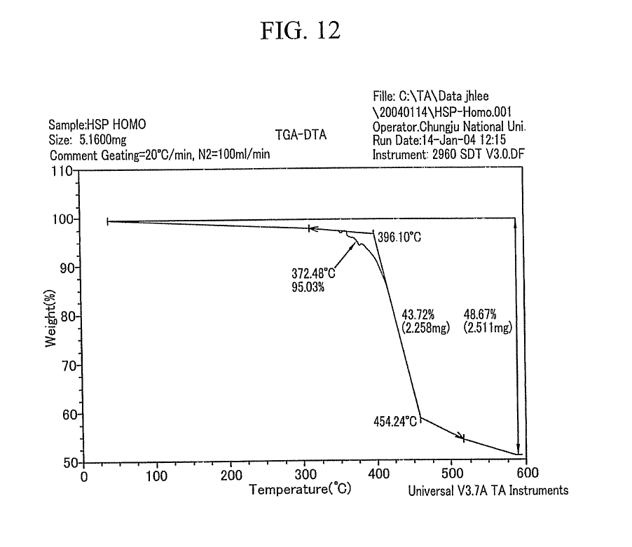

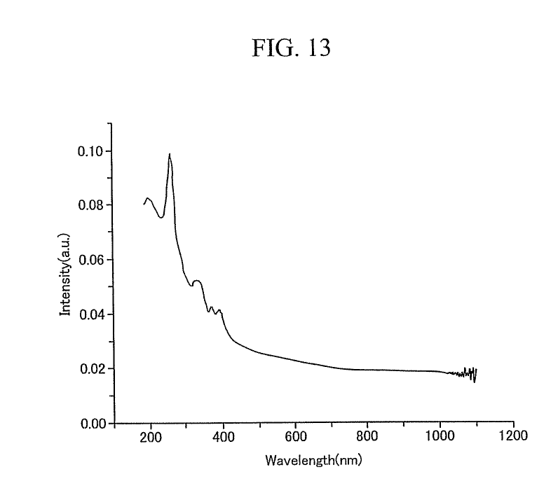

Polymers 1 and 2 synthesized in Examples 25 to 26 were analyzed regarding the thermal characteristics through TGA and DSC. The TGA analysis result regarding polymer 1 is provided in FIG. 11, and the one regarding polymer 2 was provided in FIG. 12. As shown in FIGS. 11 and 12, polymers 1 and 2 turned out to be stable near to 400° C. However, two polymers did not show any thermal transfer phenomenon up to 300° C. in DSC.

Evaluation of Optical Characteristics of Spiro-Compounds

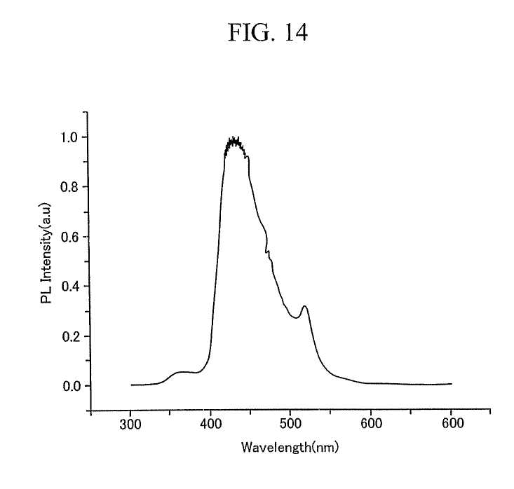

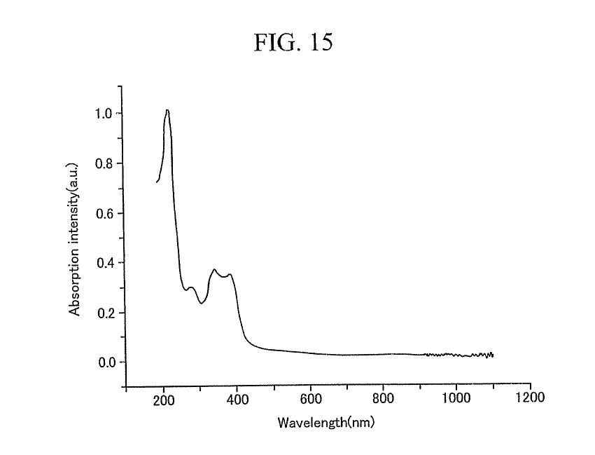

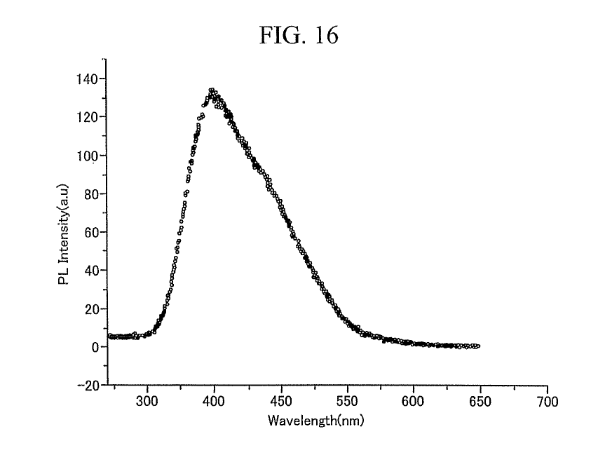

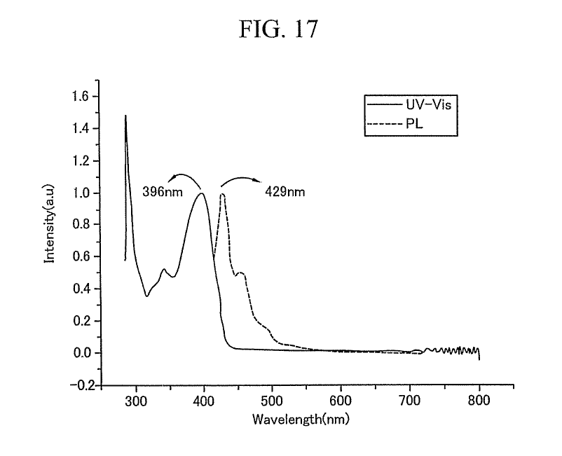

Compound 12 of Example 3 and compound 13 of Example 4 were individually dissolved in toluene. The solution was coated on a quartz substrate in a spin coating method to form a thin membrane. Then, an UV-vis spectrum and a PL (photoluminescence) spectrum were estimated regarding the membranes. The UV-vis spectra of compounds 12 and 13 are respectively provided in FIGS. 13 and 15. FIGS. 14 and 16 respectively show PL spectra of compounds 12 and 13.

As shown in FIG. 13, compound 12 had a maximum UV absorption peak at 262 nm. As shown in FIG. 14, compound 12 had a PL peak at 433 nm when the maximum absorption wavelength of compound 12 was regarded as an excitation wavelength. In addition, as shown in FIG. 15, compound 13 had a maximum UV absorption peak at 224 nm. As shown in FIG. 16, it had a maximum PL peak at about 449 nm when the maximum absorption wavelength of compound 13 was regarded as an excitation wavelength.

Polymers 1 to 3 according to Examples 25 to 27 were respectively dissolved in toluene. The solution was spin-coated in on a quartz substrate to form a polymer thin film. Then, it was measured regarding UV-vis spectrum and PL (photoluminescence) spectrum. The measurement result regarding polymer 1 is provided in FIG. 17. As shown in FIG. 17, polymer 1 had a maximum UV absorption peak at 396 nm. Considering the maximum absorption wavelength as an excitation wavelength, it had a maximum PL peak at about 429 nm. In addition, polymer 2 had a maximum UV absorption peak at 380 nm. Considering the maximum absorption wavelength as an excitation wavelength, it had a maximum PL peak at about 484 nm.

Fabrication of an Electroluminescence Display Device and Evaluation of its Characteristics

Electroluminescence display devices were fabricated by using compound 12 according to Example 3 and polymers 1 to 3 according to Examples 25 to 27, respectively.

First of all, a transparent electrode substrate, which is formed by coating ITO (indium-tin oxide) on a glass substrate, was washed. Then, the ITO was patterned to have a predetermined pattern by using a photoresist resin and etchant, and thereafter, washed again.

Next, Batron P 4083 made from Bayer Co. was coated thereon as a conductive buffer layer, and thereafter baked at 140° C. for about one hour.

An organic electroluminescence polymer solution, which was dissolved in chlorobenzene or toluene, was coated on the buffer layer by a spin coating method, and thereafter baked in a vacuum oven to completely remove a solvent, forming a compound membrane. When the polymer solution was coated by the spin coating method, it was filtered through a 0.2 mm-filter. The thickness of the compound membrane was regulated through the concentration of the solution and spinning speed. The compound membrane had a thickness ranging from about 50 to 100 nm.

Then, a Ca—Al metal electrode was vacuum-deposited on the luminescent compound membrane, maintaining less than 4×10⁻⁶ torr of a vacuum degree. Herein, a membrane thickness and a membrane growth speed were regulated by using a crystal sensor. It had 6 mm² of a luminescent area, and a forward bias voltage, which was a direct current voltage, was used as its driving voltage.

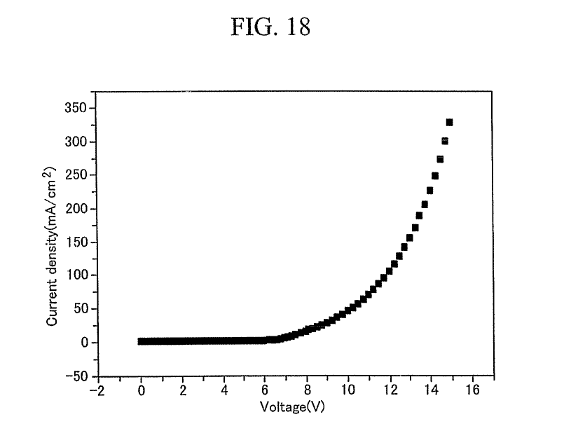

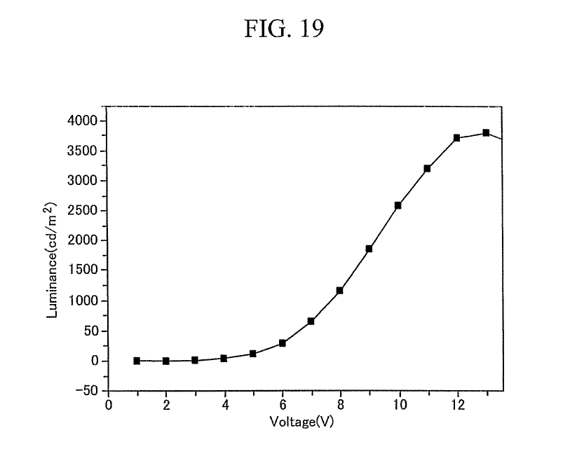

The aforementioned electroluminescence display device was formed as a single layer by forming ITO/PEDOT (poly (3,4-ethylenedioxy thiophene))/a compound12/Ca/Al in order. The compound 12 according to Example 3 and polymers 1 to 3 according to Examples 25 to 27 were examined regarding the electroluminescence characteristics. Their current-voltage graph is provided in FIG. 18, and their voltage-luminance graph is provided in FIG. 19.

The electroluminescence display devices all revealed rectifying diode characteristics.

The devices started from about 3.2 to 3.4 V of a turn-on voltage. Their luminescent color was blue, and their maximum luminance was about 3800 cd/m². Their quantum efficiency was 2.23 cd/A. In addition, after the electroluminescence display devices were repeatedly operated several times, they maintained a first voltage-current density characteristic, securing safety.

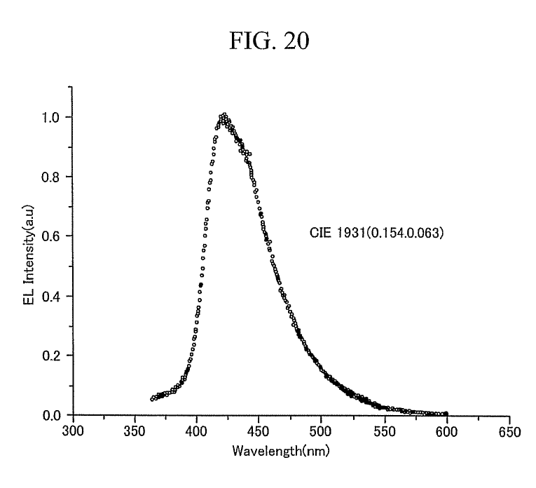

An EL spectrum of compound 12 is provided in FIG. 20. As shown in FIG. 15, compound 12 had an excellent CIE 1931 color coordinate at (151, 0.106) and respectively a maximum luminescent wavelength at 434 nm. Furthermore, even if its voltage was increased, it had the same luminescent wavelength.

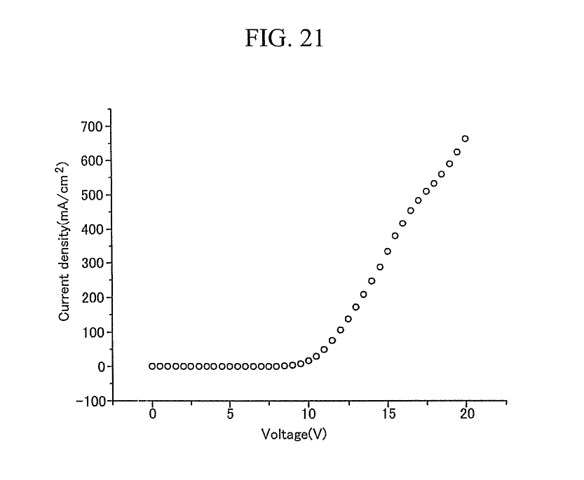

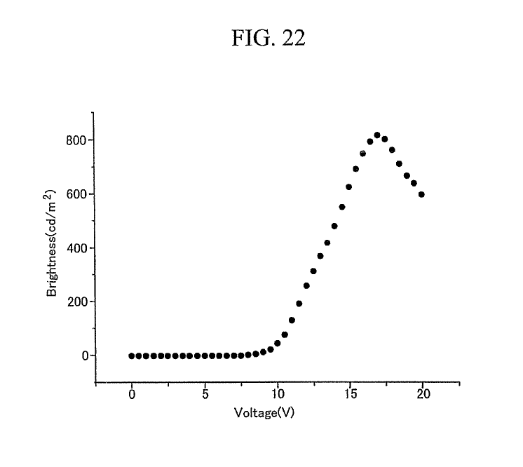

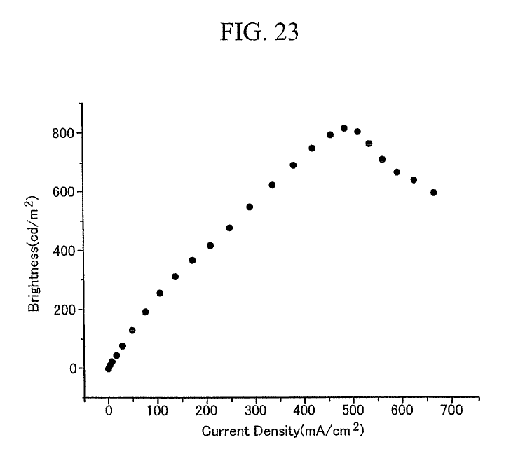

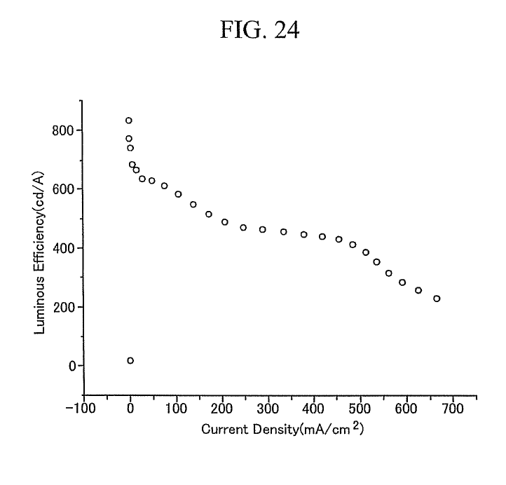

Polymer 1 was examined regarding the light emitting characteristics, and the results are provided in FIGS. 21 to 23. The electroluminescence display devices all revealed typical rectifying diode characteristics. Each device had a turn-on voltage at about 5.5 to 7.5 V (refer to FIG. 21). Its light emitting color was blue (FIG. 22), its maximum luminance was in a range of 300 to 1000 cd/m² (FIG. 23), and its maximum quantum efficiency was in a range of 0.1 to 0.34 cd/A (FIG. 24). In addtion, the electroluminescence display devices had stability of maintaining initial voltage-current density characteristic after they were repeatedly operated several times. Refering to a CIE 1931 color coordinate, polymer 1 was located at (0.21, 0.22), and polymer 3 at (0.16, 0.07), which is an excellent color coordinate that is, deep blue.

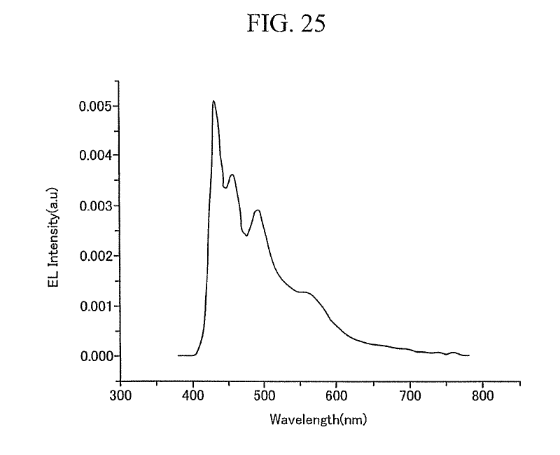

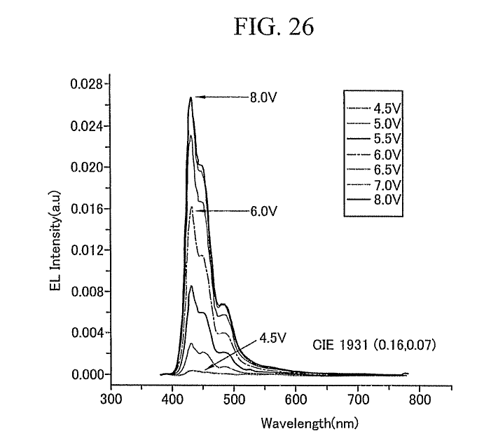

The EL spectrum of polymer 1 is illustrated in FIG. 25, and the EL spectrum of polymer 3 is illustrated in FIG. 26. As shown in FIGS. 25 and 26, polymers 1 and 3 had a maximum light emitting wavelength respectively at 431 nm and 432 nm. In addition, even when a voltage range was changed from the result of FIG. 26, its light emitting wavelength was not changed.

According to the embodiment of the present invention, a spiro-compound for an electroluminescence display device can be applied to at least one or all of a hole transport layer (HTL), a hole injection layer (HIL), an electroluminescent layer, an electron injection layer (EIL), and an electron transport layer (ETL).

Accordingly, an electroluminescence display device including the spiro-compound can realize various colors with low energy, emit blue light even at a low voltage, and have an advantage of excellently increasing luminance and luminous efficiency.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A spiro-compound for an electroluminescence display device that comprises at least one selected from the group consisting of a compound represented as the following Formulae 1 and 2, 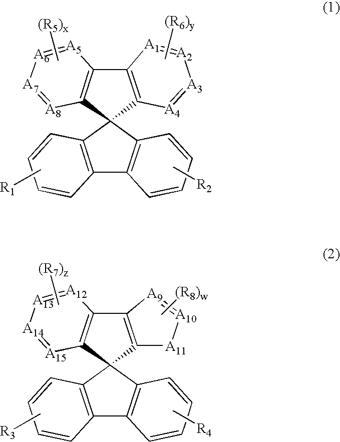
wherein, in the above formulae:
A₁ to A₁₅ are elements independently selected from the group consisting of C, N, O, S and Si, herein, at least one among A₁ to A₈ and at least one among A₉ to A₁₅ are not C;
R₁ to R₄ are independently selected from the group consisting of hydrogen, deuterium, halogen, a substituted or unsubstantiated linear or branched alkyl, a substituted or unsubstantiated cycloalkyl, a substituted or unsubstantiated alkenyl, a substituted or unsubstantiated alkynyl, a substituted or unsubstantiated alkoxy, a substituted or unsubstantiated aryl, and a substituted or unsubstituted heteroaryl;
R₅ to R₈ are selected from the group consisting of hydrogen, deuterium, halogen, a substituted or unsubstantiated linear or branched alkyl, a substituted or unsubstantiated cycloalkyl, a substituted or unsubstituted alkenyl, a substituted or unsubstituted alkynyl, a substituted or unsubstituted alkoxy, a substituted or unsubstituted aryl, a substituted or unsubstituted heteroaryl, CN, NO₂, a substituted or unsubstituted fluoroalkyl, —SiR₉R₁₀R₁₁, —NR₁₂R₁₃, and —CR₁₄═CR₁₅—R₁₆;
R₉ to R₁₆ are independently selected from the group consisting of hydrogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, and a substituted or unsubstituted heteroaryl; and
x, y, z, and w are respectively integers ranging from 0 to 4.

2. The spiro-compound of claim 1, wherein, in the R₁ to R₁₆, the alkyl is a C1 to C12 alkyl, the cycloalkyl is a C3 to C12 cycloalkyl, the alkenyl is a C2 to C8 alkenyl, the alkynyl is a C2 to C4 alkynyl, the alkoxy is a C1 to C12 alkoxy, the aryl is C4 to C30 aryl, the heteroaryl is a C4 to C30 heteroaryl including an aromatic ring including a 1 to 3 heteroatom of N, S, P, Si or O, and
the substitutents mean that at least one hydrogen is substituted with an alkyl, a cycloalkyl, an alkoxy, an alkenyl, an alkynyl, an aryl, a heteroaryl, halogen, aliphatic amine, aromatic amine, or an aryloxy.

3. The spiro-compound of claim 1, wherein, in the R₁ to R₁₆, a substituted or unsubstituted heteroaryl comprises substituted or unsubstituted cabazole, substituted or unsubstituted phenothiazine, substituted or unsubstituted phenoxazine, substituted or unsubstituted phenoxathin, substituted or unsubstituted acridine, substituted or unsubstituted phenazasiline, or substituted or unsubstituted 9-aza-10-germa-anthracene.

4. A spiro-compound for an electroluminescence display device, which comprises at least one selected from the group consisting of a compound represented as the following Formulae 3 and 4, 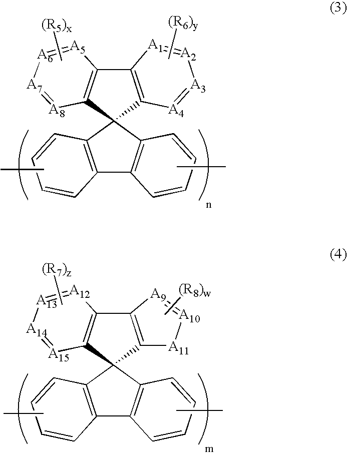
wherein, in the above formulae, A₁ to A₁₅ are an element independently selected from the group consisting of C, N, O, S, and Si, and at least one among A₁ to A₈ and at least one among A₉ to A₁₅ are not C,
R₁ to R₄ are independently selected from the group consisting of hydrogen, deuterium, halogen, a substituted or unsubstantiated linear or branched alkyl, a substituted or unsubstantiated cycloalkyl, a substituted or unsubstantiated alkenyl, a substituted or unsubstantiated alkynyl, a substituted or unsubstantiated alkoxy, a substituted or unsubstantiated aryl, and a substituted or unsubstituted heteroaryl;
R₅ to R₈ are selected from the group consisting of hydrogen, deuterium, halogen, a substituted or unsubstantiated linear or branched alkyl, a substituted or unsubstantiated cycloalkyl, a substituted or unsubstituted alkenyl, a substituted or unsubstituted alkynyl, a substituted or unsubstituted alkoxy, a substituted or unsubstituted aryl, a substituted or unsubstituted heteroaryl, CN, NO₂, a substituted or unsubstituted fluoroalkyl, —SiR₉R₁₀R₁₁, —NR₁₂R₁₃, and —CR₁₄═CR₁₅—R₁₆;
R₉ to R₁₆ are independently selected from the group consisting of hydrogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, and a substituted or unsubstituted heteroaryl;
x, y, z, and w are respectively integers ranging from 0 to 4, and
n is in a range of 1 to 10000.

5. A compound for an electroluminescence display device prepared by copolymerizing a first monomer selected from a group consiting of a compound of the following Formula 1, another compound of the following Formula 2, and a mixture thereof and a second monomer selected from a group consiting of a compound of the following Formula 5, another compound of the following Formula 6, and a mixture thereof: 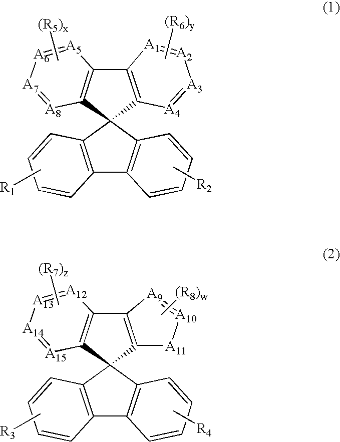
wherein, in the above formula, A₁ to A₁₅ are an element independently selected from the group consisting of C, N, O, S, and Si, and at least one among A₁ to A₈ and at least one among A₉ to A₁₅ at least are not C,
R₁ to R₄ are independently selected from the group consisting of hydrogen, deuterium, halogen, a substituted or unsubstantiated linear or branched alkyl, a substituted or unsubstantiated cycloalkyl, a substituted or unsubstantiated alkenyl, a substituted or unsubstantiated alkynyl, a substituted or unsubstantiated alkoxy, a substituted or unsubstantiated aryl, and a substituted or unsubstituted heteroaryl;
R₅ to R₈ are selected from the group consisting of hydrogen, deuterium, halogen, a substituted or unsubstantiated linear or branched alkyl, a substituted or unsubstantiated cycloalkyl, a substituted or unsubstituted alkenyl, a substituted or unsubstituted alkynyl, a substituted or unsubstituted alkoxy, a substituted or unsubstituted aryl, a substituted or unsubstituted heteroaryl, CN, NO₂, a substituted or unsubstituted fluoroalkyl, —SiR₉R₁₀R₁₁, —NR₁₂R₁₃, and —CR₁₄═CR₁₅—R₁₆;
R₉ to R₁₆ are independently selected from the group consisting of hydrogen, a substituted or unsubstituted linear or branched alkyl, a substituted or unsubstituted cycloalkyl, a substituted or unsubstituted alkenyl, a substituted or unsubstituted alkynyl, a substituted or unsubstituted alkoxy, a substituted or unsubstituted aryl, and a substituted or unsubstituted heteroaryl;
x, y, z, and w are indepencantly an integer ranging from 0 to 4: 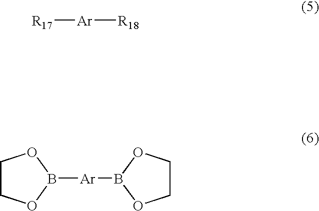
wherein, in the above formulae, Ar is an aromatic group or a heteroaromatic group including more than one heteroatom in an aromatic ring, R₁₇ and R₁₈ are selected from the group consisting of a reactive functional group, hydrogen, unsubstituted linear or branched alkyl, cycloalkyl, alkenyl, alkynyl, alkoxy, an aryl, and heteroaryl.

6. The compound of claim 5, which includes the first monomer and the second monomer in a mole ratio ranging 1:0.01 to 100.

7. An electroluminescence display device comprising:
a substrate;
an anode;
a hole injection layer (HIL);
a hole transport layer (HTL);
an electroluminescent layer;
an electron transport layer (ETL); and
a cathode, wherein, at least one of the hole injection layer (HIL), the hole transport layer (HTL), the electroluminescent layer, and the electron transport layer (ETL) comprises a spiro-compound according to claim 1.

8. The electroluminescence display device of claim 7, wherein the electroluminescent layer comprises the spiro-compound.

9. The electroluminescence display device of claim 8, wherein the electroluminescent layer further comprises a dopant that has less energy gap than a spiro-compound and a conjugated double bond.

10. The electroluminescence display device of claim 9, wherein the dopant is selected from the group consisting of dicarbazolylazobenzene (DCAB), fluorenyldiacetylene (FDA), perylene, carbazole, carbazolederivative, a coumarin-based compound, and 4-(dicyanomethylene)-2-methyl-6-(1,1,7,7-tetramethyljulodinyl-9-enyl)-4H-pyran (DCJT).

11. The electroluminescence display device of claim 7, which further comprises a buffer layer between the anode and the hole injection layer (HIL).

12. The electroluminescence display device of claim 7, which further comprises an electron injection layer (EIL) between the electron transport layer (ETL) and the cathode, and
wherein at least one of the hole injection layer (HIL), the hole transport layer (HTL), the electroluminescent layer, the electron transport layer (ETL), and the electron transport layer (ETL) comprises a spiro-compound according to claim 1.
